(19) United States
(12) Patent Application Publication
(10) Pub. No.: US 2012/0207687 A1
(43) Pub. Date: Aug. 16, 2012

(54) TOPICAL FORMULATIONS OF TARGETED NITROXIDE AGENTS

(75) Inventors: Louis D. Falo, JR., Wexford, PA (US); Joel S. Greenberger, Sewickley, PA (US); Peter Wipf, Pittsburgh, PA (US)

(73) Assignee: University of Pittsburgh-Of the Commonwealth System of Higher Education

(21) Appl. No.: 13/349,526

(22) Filed: Jan. 12, 2012

Related U.S. Application Data
(60) Provisional application No. 61/433,111, filed on Jan. 14, 2011.

(51) Int. Cl. A61K 31/445; A61P 17/00; A61Q 17/04; A61K 8/49

(52) U.S. Cl. 424/59; 514/315

(57) ABSTRACT

A method for preventing or treating skin damage in a radiotherapy subject, comprising topically administering to the subject a composition that includes a therapeutically effective amount at least one targeted nitroxide agent and at least one additional ingredient.

### PRIORITY CLAIM

This application claims the benefit of U.S. Provisional Application No. 61/433,111, filed Jan. 14, 2011, which is incorporated herein by reference in its entirety.

### ACKNOWLEDGMENT OF GOVERNMENT SUPPORT

This invention was made with government support under grant number CA081284 awarded by the National Institutes of Health, Department of Health and Human Services. The government has certain rights in the invention.

### BACKGROUND

The biologic consequences of exposure to ionizing radiation (IR) include genomic instability and cell death (Little J B, Nagasawa H, Pfenning T, et al. Radiation-induced genomic instability: Delayed mutagenic and cytogenetic effects of X rays and alpha particles. Radiat Res 1997; 148:299-307). It is assumed that radiolytically generated radicals are the primary cause of damage from IR. Direct radiolysis of water and the secondary reactive intermediates with a short lifetime (10⁻¹⁰-10⁻⁶ seconds) mediate the chemical reactions that trigger the damage of cellular macromolecules, including DNA and proteins, as well as phospholipids in membranes (Mitchell J B, Russo A, Kuppusamy P, et al, Radiation, radicals, and images. Ann N Y Acad Sci 2000; 899:28-43). The DNA is believed to be the primary target for the radical attack, resulting in single and double DNA strand breaks (Bryant P E. Enzymatic restriction of mammalian cell DNA: Evidence for double-strand breaks as potentially lethal lesions. Int J Radiat Biol 1985; 48:55-60). To maintain the genomic integrity, multiple pathways of DNA repair and cell-cycle checkpoint control are activated in response to irradiation-induced DNA damage (Elledge S J. Cell cycle checkpoints: Preventing an identity crisis. Science 1996; 274:1664-1672). Failure of these repair and regulatory systems leads to genotoxicity, malignant transformation, and cell death (Sachs R K, Chen A M, Brenner D J. Proximity effects in the production of chromosome aberrations by ionizing radiation. Int J Radiat Biol 1997; 71:1-19).

One of the major mechanisms of IR-induced cell death is apoptosis, most commonly realized through a mitochondria-dependent intrinsic pathway (Newton K, Strasser A. Ionizing radiation and chemotherapeutic drugs induce apoptosis in lymphocytes in the absence of Fas or FADD/MORT1 signaling. Implications for cancer therapy. J Exp Med 2000; 191:195-200). The latter includes permeabilization of mitochondria followed by the release of cytochrome (cyt) c and other proapoptotic factors (Smac/Diablo [second mitochondrial-derived activator of caspase/direct inhibitor of apoptosis-binding protein with low pI], EndoG [endonuclease G], Omi/HtrA2, and AIF [apoptosisinducing factor]) into the cytosol as the key events in the execution of the death program. The released cyt c facilitates the formation of apoptosomes by interacting with apoptotic protease activating factor 1 (Apaf-1) and then recruits and activates procaspase-9 and triggers the proteolytic cascade that ultimately leads to cell disintegration. Release of proapoptotic factors and caspase activation designate the commencement of irreversible stages of apoptosis. Therefore, significant drug discovery efforts were directed toward the prevention of these events, particularly of the mitochondrial injury representing an important point of no return (Szewczyk A, Wojtczak L. Mitochondria as a pharmacological target. Pharmacol Rev 2002; 54:101-127). However, the exact mechanisms of cyt c release from mitochondria are still poorly understood. It was postulated that generation of reactive oxygen species (ROS), likely by means of disrupted electron transport, has a crucial role in promoting cyt c release from mitochondria (Kowaltowski A J, Castilho R F, Vercesi A E. Opening of the mitochondrial permeability transition pore by uncoupling or inorganic phosphate in the presence of Ca2+ is dependent on mitochondrial-generated reactive oxygen species, FEBS Lett 1996; 378:150-152). Notably, ROS can induce mitochondria membrane permeabilization both in vitro and in vivo, and the mitochondrial membrane transition pore was shown to be redox sensitive (Kroemer G, Reed J C. Mitochondrial control of cell death. Nat Med 2000; 6:513-519).

Conversely, antioxidants and reductants, overexpression of manganese superoxide dismutase (MnSOD) (Wong G H, Elwell J H, Oberley L W, et al. Manganous superoxide dismutase is essential for cellular resistance to cytotoxicity of tumor necrosis factor. Cell 1989; 58:923-931), and thioredoxin (Iwata S, Hori T, Sato N, et al. Adult T cell leukemia (ATL)-derived factor/human thioredoxin prevents apoptosis of lymphoid cells induced by L-cystine and glutathione depletion: Possible involvement of thiol-mediated redox regulation in apoptosis caused by pro-oxidant state. J Immunol 1997; 158:3108-3117) can delay or inhibit apoptosis. Previous studies showed that early in apoptosis, a mitochondria-specific phospholipid-cardiolipin (CL) translocated from the inner to the outer mitochondrial membrane and activated cyt c into a CL-specific peroxidase (Fernandez M G, Troiano L, Moretti L, et al. Early changes in intramitochondrial cardiolipin distribution during apoptosis. Cell Growth Differ 2002; 13:449-455 and Kagan V E, Tyurin V A, Jiang J, et al. Cytochrome c acts as a cardiolipin oxygenase required for release of proapoptotic factors. Nat Chem Biol 2005; 1:223-232). The activated cyt c further catalyzed the oxidation of CL by using mitochondrially generated ROS (Kagan V E, Tyurin V A, Jiang J, et al. Cytochrome c acts as a cardiolipin oxygenase required for release of proapoptotic factors. Nat Chem Biol 2005; 1:223-232). Most importantly, oxidized CL is an important contributor to the release of cyt c from mitochondria (Kagan V E, Tyurin V A, Jiang J, et al. Cytochrome c acts as a cardiolipin oxygenase required for release of proapoptotic factors. Nat Chem Biol 2005; 1:223-232 and Petrosillo G, Casanova G, Matera M, et al. Interaction of peroxidized cardiolipin with rat-heart mitochondrial membranes: Induction of permeability transition and cytochrome c release. FEBS Lett 2006; 580:6311-6316), which might be attributed to changes in microenvironment for the interaction between this phospholipid and cyt c (Ott M, Robertson J D, Gogvadze V, et al. Cytochrome c release from mitochondria proceeds by a two-step process. Proc Natl Acad Sci U S A 2002; 99:1259-1263 and Gamido C, Galluzzi L, Brunet M, et al. Mechanisms of cytochrome c release from mitochondria. Cell Death Differ 2006; 13:1423-1433) and/or participation of oxidized CL in the formation of mitochondrial permeability transition pores (MTP) in coordination with Bcl-2 family proteins (Bid, Bax/Bak), as well as adenine nucleotide translocator (ANT) and voltage-dependent anion channel (VDAC) (Petrosillo G, Casanova G, Matera M, et al. Interaction of peroxidized cardiolipin with rat-heart mitochondrial membranes: Induction of permeability transition and cytochrome c release. FEBS Lett 2006; 580:6311-6316 and Gonzalvez F, Gottlieb E. Cardiolipin: Setting the beat of apoptosis. Apoptosis 2007; 12:877-885). In addition to their essential role in the apoptotic signaling pathway, ROS were also implicated in perpetuation of the bystander effect (Narayanan P K, Goodwin E H, Lehnert B E. Alpha particles initiate biological production of superoxide anions and hydrogen peroxide in human cells. Cancer Res 1997; 57:3963-3971 and Iyer R, Lehnert B E. Factors underlying the cell growth-related bystander responses to alpha particles. Cancer Res 2000; 60:1290-1298) and genomic instability after irradiation exposure (Spitz D R, Azzam E I, Li J J, et al. Metabolic oxidation/reduction reactions and cellular responses to ionizing radiation: A unifying concept in stress response biology, Cancer Metastasis Rev 2004; 23:311-322; Limoli C L, Giedzinski E, Morgan W F, et al. Persistent oxidative stress in chromosomally unstable cells. Cancer Res 2003; 63:3107-3111; and Kim G J, Chandrasekaran K, Morgan W F. Mitochondrial dysfunction, persistently elevated levels of reactive oxygen species and radiation-induced genomic instability: A review. Mutagenesis 2006; 21:361-367). Hence, elimination of intracellular ROS, particularly its major source, mitochondrial ROS, by antioxidants may be an important opportunity for developing radioprotectors and radiomitigators. Protection by antioxidants against IR has been studied for more than 50 years (Weiss J F, Landauer M R. Radioprotection by antioxidants. Ann N Y Acad Sci 2000; 899:44-60).

One of the major mechanisms of ionizing irradiation induced cell death is apoptosis, most commonly realized through a mitochondria dependent intrinsic pathway. Oxidation of cardiolipin catalyzed by cytochrome c, release of cytochrome c and other pro-apoptotic factors into the cytosol and subsequent caspase activation are the key events in the execution of the death program designating the commencement of irreversible stages of apoptosis.

In Belikova, N A, et al., (Cardiolipin-Specific Peroxidase Reactions of Cytochrome C in Mitochondria During Irradiation-Induced Apoptosis, Int. J. Radiation Oncology Biol. Phys 2007, 69(1): 176-186), a small interfering RNA (siRNA) approach was used to engineer HeLa cells with lowered contents of cyt c (14%, HeLa 1.2 cells). Cells were treated by γ-irradiation (in doses of 5-40 Gy). Lipid oxidation was characterized by electrospray ionization mass spectrometry analysis and fluorescence highperformance liquid chromatography-based Amplex Red assay. Release of a proapoptotic factor (cyt c, Smac/DIABLO) was detected by Western blotting. Apoptosis was revealed by caspase-3/7 activation and phosphatidylserine externalization. They showed that irradiation caused selective accumulation of hydroperoxides in cardiolipin (CL) but not in other phospholipids. HeLa 1.2 cells responded by a lower irradiation-induced accumulation of CL oxidation products than parental HeLa cells. Proportionally decreased release of a proapoptotic factor, Smac/DIABLO, was detected in cyt c-deficient cells after irradiation. Caspase-3/7 activation and phosphatidylserine externalization were proportional to the cyt c content in cells. They concluded that cytochrome c is an important catalyst of CL peroxidation, critical to the execution of the apoptotic program. This new role of cyt c in irradiation-induced apoptosis is essential for the development of new radioprotectors and radiosensitizers.

Significant drug discovery efforts have been directed towards prevention of these events, particularly of the mitochondrial injury that represents an important point of no return. Although the exact mechanisms are still not well understood, generation of reactive oxygen species (ROS) and oxidation of cardiolipin by the peroxidase function of cytochrome c/cardiolipin complexes are believed to play a critical role in promoting cytochrome c release from mitochondria. ROS-superoxide radicals dismutating to H₂O₂-feed the peroxidase cycle and facilitate accumulation of oxidized cardiolipin. Hence, elimination of intracellular ROS, particularly its major source, mitochondrial ROS, by electron and radical scavengers is a promising opportunity for developing radioprotectors and radiomitigators. Significant research has been conducted in the field of radiation protection to use antioxidants against ionizing irradiation (Weiss et al. Radioprotection by Antioxidants. Ann N Y Acad Sci 2000; 899:44-60).

A new class of antioxidants, stable nitroxide radicals, has been suggested as potent radioprotectors due to multiplicity of their direct radical scavenging properties as well as catalytic enzyme-like mechanisms (Saito et al. Two reaction sites of a spin label, TEMPOL with hydroxyl radical. J Pharm Sci 2003; 92:275-280; Mitchell et al. Biologically active metal-independent superoxide dismutase mimics. Biochemistry 1990; 29:2802-2807). TEMPOL (4-hydroxy-2,2,6,6-tetramethylpiperidine-N-oxyl) is a nitroxide whose properties as a radioprotector in vitro and in vivo have been extensively studied (Mitchell et al. Nitroxides as radiation protectors. Mil Med 2002; 167:49-50; Hahn et al. In vivo radioprotection and effects on blood pressure of the stable free radical nitroxides. Int J Radiat Oncol Biol Phys 1998; 42:839-842. Mitchell et al. Inhibition of oxygen-dependent radiation-induced damage by the nitroxide superoxide dismutase mimic, tempol. Arch Biochem Biophys 1991; 289:62-70; Hahn et al. Tempol, a stable free radical, is a novel murine radiation protector. Cancer Res 1992; 52:1750-1753). Currently, TEMPOL is in clinical trials as a topical radiation protector to prevent hair loss during cancer radiotherapy. While found promising and relatively effective, the required high millimolar concentrations of TEMPOL, mainly due to its poor partitioning into cells and mitochondria, set a limit for its broader applications (Gariboldi et al. Study of in vitro and in vivo effects of the piperidine nitroxide Tempol—a potential new therapeutic agent for gliomas. Eur J Cancer 2003; 39:829-837). In addition, it has been demonstrated that TEMPOL must be present during irradiation to exert its radioprotective effect (Mitchell et al. Radiation, radicals, and images. Ann N Y Acad Sci 2000; 899:28-43; Mitchell et al. Inhibition of oxygen-dependent radiation-induced damage by the nitroxide superoxide dismutase mimic, tempol. Arch Biochem Biophys 1991; 289:62-70), This suggests that the protective mechanisms of TEMPOL are limited to its interactions with short-lived radiolytic intermediates produced by irradiation.

Sufficient concentrations of antioxidants at the sites of free radical generation are critical to optimized protection strategies. A great deal of research has indicated that mitochondria are both the primary source and major target of ROS (Reviewed in Orrenius S. Reactive oxygen species in mitochondria-mediated cell death. Drug Metab Rev 2007; 39:443-455). In fact, mitochondria have been long considered as an important target for drug discovery (Szewczyk et al., Mitochondria as a pharmacological target. 221 Pharmacol. Rev. 54:101-127; 2002; Garber K. Targeting mitochondria emerges as therapeutic strategy. J. Natl. Cancer Inst. 97:1800-1801; 2005).

Chemistry-based approaches to targeting of compounds to mitochondria include the use of proteins and peptides, as well as the attachment of payloads to lipophilic cationic compounds, triphenyl phosphonium phosphate, sulfonylureas, anthracyclines, and other agents with proven or hypothetical affinities for mitochondria (Murphy M P. Targeting bioactive compounds to mitochondria. Trends Biotechnol. 15:326-330; 1997; Dhanasekaran et al., Mitochondria superoxide dismutase mimetic inhibits peroxideinduced oxidative damage and apoptosis: role of mitochondrial superoxide. Free Radic. Biol. Med. 157 39:567-583; 2005; Hoye et al, Targeting Mitochondria. Acc. Chem. Res. 41: 87-97, 2008).

One type of radiation exposure is radiotherapy. Radiation therapy is an important tool in the fight against cancer and is used in the treatment of as many as 50% of all cancer patients. Accordingly, more than half a million cancer patients receive radiation therapy each year. While the use of radiation therapy is an effective way to treat many kinds of cancer, there are many complications that may result. Common complications can include negative effects on the patient's skin, hair follicles, and mucous membranes.

Common skin complications of radiotherapy include erythema and folliculitis. These disorders can be very irritating to patients as they both involve pruritus and redness of the skin. These and other skin complications can arise through oxidative and other stress caused by radiation. Other examples of skin conditions caused by radiation include fibrosis, dry desquamation and moist desquamation. For instance, exposure to high doses of ionizing electron beam irradiation results in a significant skin burn.

In addition, hair follicles are quite sensitive to radiotherapy. Accordingly, if hair is in the radiation treatment beam field, it can cease to grow and fall out. Losing one's hair can be a source of embarrassment and loss of self esteem.

Radiotherapy can also have negative effects on the mucous membranes in the eyes, nose, mouth, vagina, rectal mucosa and the like. For example, oral mucositis, also called stomatitis, results from the local effects of radiation to the oral mucosa. Mucositis is characterized by inflammation of the mucosa of the mouth and ranges from redness to severe ulceration. Symptoms of mucositis vary from pain and discomfort, to an inability to tolerate food or fluids. Even worse, oral mucositis may be so severe as to limit the patient's ability to tolerate further radiotherapy or chemotherapy.

Patients with damaged oral mucosa and a reduced immunity resulting from radiotherapy are also prone to opportunistic infections in the mouth. Accordingly, mucositis may also further compromise a patient's response to treatment and/or palliative care. It is therefore extremely important that mucositis be prevented whenever possible, or at least treated to reduce its severity and possible complications.

Another common mucous membrane condition caused by radiotherapy is proctitis. Proctitis is an inflammation of the lining of the rectum (rectal mucosa). The most common symptom is a frequent, or continuous sensation, or urge to have a bowel movement. Other symptoms include constipation, a feeling of rectal fullness, left-sided abdominal pain, passage of mucus through the rectum, rectal bleeding, and anorectal pain.

Finally, even if the radiation induced damage is sublethal, long term damage to soft tissues, such as fibrosis, can be very debilitating. In addition, mutagenic lesions can have serious long term consequences, including carcinogenesis.

Another source of radiation is ultraviolet (UV) radiation, typically from sun exposure. It is known that the human epidermis can be tanned by light radiation having a wavelength in the range from 280 to 400 nm and that radiation having a wavelength in the range from 280 to 320 nm, which is known under the term UV-B, causes erythema and skin burning, which may be detrimental to the formation of a natural tan. The UV-B radiation should therefore be filtered out.

It is furthermore known that UV-A radiation having a wavelength in the range from 320 to 400 nm, which tans the skin, can cause a change in the skin, in particular in the case of sensitive skin or skin which is exposed continuously to sunlight. UV-A radiation causes, in particular, a loss in skin elasticity and wrinkling, which results in premature ageing. It favors the triggering of erythema formation or increases this reaction in some people, and it can even be the cause of toxic or allergic reactions triggered by light. It is therefore desirable also to filter out the UV-A radiation.

Oxidative damage is a critical final common pathway in skin damage resulting from a variety of radiation (including UVA and UVB) and toxin exposures that can result in a broad range of unwanted events including unwanted cosmetic effects (skin aging and wrinkling) carcinogenesis (including melanoma and squamous and basal cell carcinomas), and radiation dermatitis.

Another radiation exposure event is the need to assuage the consequences of unplanned irradiation associated with civil scenarios, such as radiation accidents and radiation terrorism, as well as irradiation in military contexts. Cutaneous injury from ionizing irradiation and combined thermal/ionizing radiation damage is a significant component of the tissue injury from radiological dispersion devices and fission bomb terrorist events. In particular beta particle radiation from isotopic fallout irradiation produces significant cutaneous injury. Dermal erythema and ulceration can result in wound infection followed by systemic sepsis. Common measures currently utilized to treat ionizing irradiation/thermal burns rely heavily on experience with thermal burn injury.

### SUMMARY

One embodiment disclosed herein is directed to a method for preventing or treating skin damage in a radiotherapy subject, comprising topically administering to the subject a composition that includes a therapeutically effective amount at least one targeted nitroxide agent and at least one additional ingredient.

A further embodiment disclosed herein is directed to a method for preventing or treating UV-induced damage in a subject, comprising topically administering to the subject a composition that includes at least one targeted nitroxide agent and at least one additional ingredient, wherein the targeted nitroxide agent is present in the composition in an amount sufficient to achieve a cutaneous cumulative concentration of the targeted nitroxide agent in the subject of 1 μmole/mg to 100 μmole/mg over a 24 hour period after administration.

Another embodiment disclosed herein is directed to a composition for topically administering to a subject, wherein the composition comprises 0.1 to 100 mg/ml of at least one targeted nitroxide agent and at least one additional ingredient; the composition is in the form of a suspension, colloid or emulsion; and the composition has a sufficient viscosity that keeps the targeted nitroxide agent in contact with a treated area for a sufficient period of time to allow suitable absorption into the treated area.

The foregoing will become more apparent from the following detailed description, which proceeds with reference to the accompanying figures.

### BRIEF DESCRIPTION OF THE DRAWINGS

FIG. 1 provides non-limiting examples of certain nitroxides. The log P values were estimated using the online calculator of molecular properties and drug likeness on the Molinspirations Web site (www.molinspiration.com/cgi-bin/properties). TIPNO=tert-butyl isopropyl phenyl nitroxide.

FIG. 2 provides examples of structures of certain mitochondria-targeting antioxidant compounds referenced herein, and the structure of TEMPOL.

FIG. 3 depicts an example of a synthetic pathway for the TEMPO-hemigramicidin conjugates.

FIG. 4A is a schematic of a synthesis protocol for JP4-039. FIG. 4B provides a synthesis route for a compound of Formula 4, below.

FIGS. 5A and 5B provide structures for compounds JED-E71-37 and JED-E71-58, respectively.

FIG. 6 is a schematic showing alternative designs of nitroxide analogues.

FIG. 7 is a schematic of a synthesis protocol for various alternative designs of nitroxide analogues.

FIG. 8 is a schematic of a synthesis protocol for an alternative nitroxide moiety of 1,1,3,3-tetramethylisoindolin-2-yloxyl (TMIO).

FIG. 9 is a schematic of a synthesis protocol for an alternative nitroxide moiety of 1-methyl azaadamantane N-oxyl (1-Me-AZADO).

FIG. 10 depicts data demonstrating that topically applied JP4-039 protects human skin from oxidative skin damage as described in Example 1 below.

FIGS. 11-14 depict data demonstrating that topically applied JP4-039 is an antioxidant that can mitigate skin damage from ionizing radiation as described in Example 2 below.

FIG. 15 is a schematic representation of a Bronaugh diffusion system for studying in vitro transdermal flux.

FIG. 16 is a graph depicting delivery of XJB-5-125 into mouse skin after 24 hours.

FIG. 17 is a graph depicting cumulative transdermal absorption of XJB-5-125 through mouse skin over 24 hours.

FIG. 18 depicts data demonstrating that topically applied targeted nitroxides mitigate oxidative stress induced skin damage as described in Example 5 below.

FIG. 19 depicts data demonstrating that topically applied targeted nitroxides mitigate oxidative stress induced skin damage even when applied 24 hours after exposure to ionizing radiation.

FIG. 20 is a schematic of a synthesis protocol for another targeted nitroxide.

### DETAILED DESCRIPTION

As used herein, the singular terms “a,” “an,” and “the” include plural referents unless context clearly indicates otherwise. Also, as used herein, the term “comprises” means “includes.”

Although methods and materials similar or equivalent to those described herein can be used in the practice or testing of the present disclosure, suitable methods and materials are described below. In addition, the materials, methods, and examples are illustrative only and not intended to be limiting.

To facilitate review of the various examples of this disclosure, the following explanations of specific terms are provided:

“Administration” as used herein is inclusive of administration by another person to the subject or self-administration by the subject.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

The term “co-administration” or “co-administering” refers to administration of the targeted nitroxide agent-containing composition disclosed herein with at least one other therapeutic agent within the same general time period, and does not require administration at the same exact moment in time (although co-administration is inclusive of administering at the same exact moment in time). Thus, co-administration may be on the same day or on different days, or in the same week or in different weeks. The additional therapeutic agent may be included in the same composition as the targeted nitroxide agent or it may be included in another composition such as a sunscreen lotion.

“Composition” and “formulation” are used herein interchangeably.

“Inhibiting” refers to inhibiting the full development of a disease or condition. “Inhibiting” also refers to any quantitative or qualitative reduction in biological activity or binding, relative to a control.

The term “protects” or “protecting” refers to a reduction in the amount of skin damage which can be manifested, e.g., by a decrease in the number and/or severity of individual skin lesions, or prevention of the development of skin lesions.

A “therapeutically effective amount” refers to a quantity of a specified agent sufficient to achieve a desired effect in a subject being treated with that agent. For example, a therapeutically amount may be an amount of a targeted nitroxide agent that is sufficient to provide a bioactive amount in a subject's epidermis or dermis. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

The term “topical” as used herein refers to the route of administration of a composition that involves direct application to the body part being treated, e.g., the skin for dermatological compositions. Examples of topical application include application to the skin of gels or other semisolids to rub-on, solutions to spray, or liquids to be applied by an applicator. Rinse-off application with washes, cleansers, or shampoos are also examples of topical application. Typically, areas of the body suitable for application of compositions include the skin of the face, throat, neck, scalp, chest, back, ears, and other skin sites where sun exposure may occur.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” or “treating skin damage” refers to inhibiting the full development of a disease or a skin condition, for example, in a subject who is at risk for a disease such as cancer, particularly a metastatic cancer, or skin damage such as a radiotherapy patient. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition.

In certain embodiments disclosed herein small molecule mitochondrial targeted anti-oxidants (such as JP4-039 or analogs thereof as described below) are applied directly to human skin using an appropriate topical formulation or device. In human skin the small molecule mitochondrial targeted anti-oxidants exert powerful antioxidant and electron-scavenging functions capable of preventing or reversing deleterious oxidative-stress related damage in human skin. In one embodiment, JP4-039 (or analogs thereof as described below) can prevent and mitigate oxidative damage caused by ionizing radiation in human skin. In support of this function, we have shown that JP4-039 formulated for topical delivery to human skin, when applied to living human skin explants, potently prevents and inhibits oxidative damage as evidenced by decreasing the depletion of the antioxidant GSH, reduction of formation of protein carbonyls, reduction of radiation induced cellular apoptosis, and reduction of epidermal thickening associated with oxidative skin damage. These human skin studies demonstrate that topically applied JP04-39 is a potent antioxidant that can prevent and mitigate oxidative damage in human skin.

The radioprotective formulations are useful to prevent, mitigate, or treat radiation induced damage to cells tissues, or organs, and/or organisms, that have already been exposed to radiation (e.g., from clinical or non-clinical sources), or as prophylactics to mitigate or prevent damage to cells tissues or organs, and/or organisms that are expected to be exposed to radiation (e.g., in anticipation of radiotherapy, in certain military contexts, and the like).

### Topical Formulations

The topical formulations disclosed herein include at least one targeted nitroxide agent as described below in more detail. In certain embodiments, the targeted nitroxide agent is a mitochondria-targeted nitroxide. The topical formulations include, but are not limited to, gels, creams, lotions, solutions, hydrophilic or hydrophobic ointments, microemulsions, shake-powders, aerosol and pump sprays, foams, patches, and films.

In certain embodiments, the targeted nitroxide agent is not dissolved in a solvent but is present in the formulation as a component of a multiphase or heterogeneous mixture. The heterogeneous mixture may be, for example, a suspension, a colloid, or an emulsion. In preferred embodiments, the topical formulation is an emulsion.

The formulation may have a sufficient viscosity such that the formulation does not immediately run off the treated area upon application to a patient. In certain embodiments the pharmaceutical composition should have a viscosity that keeps the nitroxide radioprotector and other active ingredients in contact with the treated area for a sufficient period of time to allow suitable absorption to the treated area.

In certain embodiments, the amount of targeted nitroxide agent(s) present in the formulation is from 0.1 to 100 mg/ml of the formulation, more particularly 0.5 to 10 mg/ml, and most particularly 1 to 8 mg/ml. In certain embodiments, the amount of targeted nitroxide agent(s) present in the formulation is sufficient to achieve a cutaneous cumulative concentration of the targeted nitroxide agent in the subject of 1 pmole/mg to 100 μmmole/mg, more particularly 5 pmole/mg to 40 μmole/mg, and most particularly 10 pmole/mg to 20 μmole/mg, over a 24 hour period after administration of the formulation.

In some embodiments, liquids and thickened liquids can be topically applied with the aid of an applicator to allow suitable application of the targeted nitroxide agent to the treated area. Applicators can include, but are not limited to, cloths, rags, sponges, towels, gauze, and like absorbent materials, and the combination of the applicator and the formulation is one aspect of the methods described herein.

In addition to including a targeted nitroxide agent, the topical compositions disclosed herein also include at least one other ingredient which may be, but is not limited to, polymers, colorants, antimicrobials, preservatives, antioxidants, alcohols, emollients, additional active ingredients, ingredients that enhance the permeability of the treated area, water, a pharmaceutically acceptable carrier, or an excipient.

In certain embodiments, the formulation is a liposome or multiphase (a liquid comprising more than one phase, such as oil in water, water in oil, liposomes or multi-lamellar structures) composition that may include at least one additional ingredient selected from a phospholipid, a non-ionic detergent, and a cationic lipid. One example is a composition comprising a phosphatidyl choline, a non-ionic surfactant, and a quaternary ammonium salt of a lipid-substituted D or L glutamic acid or aspartic acid, and an aqueous solvent. The liposomes or multiphase liquids and the ingredients thereof are pharmaceutically acceptable. They are typically formulated using an aqueous solvent, such as water, normal saline or PBS.

Phospholipids include any natural or synthetic diacylglyceryl phospholiopids (such as phosphatidyl choline, phosphotidylethanolamine, phosphotidylserine, phosphatidylinositol, phosphatidylinositol phosphate, etc) and phosphosphingolipids that is capable of forming selfassembly liposomes. In one example the phospholipid is a phosphatidyl choline, a compound that comprises a choline head group, glycerophosphoric acid and fatty acid, Phosphatidyl choline can be obtained from eggs, soy or any suitable source and can be synthesized.

A nonionic surfactant, is a surfactant containing no charged groups. Nonionic surfactants comprise a hydrophilic head group and a lipophilic tail group, such as a single- or double-lipophilic chain surfactant. Examples of lipophilic tail groups include lipophilic saturated or unsaturated alkyl groups (fatty acid groups), steroidal groups, such as cholesteryl, and vitamin E (e.g., tocopheryl) groups, such as a polysorbate (a polyoxyethylene sorbitan), for example Tween 20, 40, 60 or 80. More broadly, non-ionic surfactants include: glyceryl esters, including mono-, di- and tri-glycerides; fatty alcohols; and fatty acid esters of fatty alcohols or other alcohols, such as propylene glycol, polyethylene glycol, sorbitan, sucrose and cholesterol.

A cationic lipid is a compound having a cationic head and a lipophilic tail. Included are cationic lipids that are quaternary ammonium salts, such as quaternary ammonium salts of lipidsubstituted D and L glutamic acid or aspartic acid, such as glutamic acid dialkyl amides, including for example L-glutamic acid-1,5,-dioleyl amide. Other commercially-available examples of cationic lipids (e.g., available from Avanti Polar Lipids) include DC-Cholesterol (3β-[N—(N,N-dimethylaminoethane)-carbamoyl]cholesterol hydrochloride), DOTAP (e.g., 1,2-dioleoyl-3-trimethylammonium-propane (chloride salt)), DODAP (e.g., 1,2-dioleoyl-3-dimethylammoniumpropane), DDAB (e.g., Dimethyldioctadecylammonium (Bromide Salt)), ethyl-PC (e.g., 1,2-dilauroylsn-glycero-3-ethylphosphocholine (chloride salt)) and DOTMA (e.g., 1,2-di-O-octadecenyl-3-trimethylammonium propane (chloride salt)).

Other formulations for topical delivery include, but are not limited to, ointments, gels, sprays, fluids, and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Those with skill in the art can readily modify the thickness of topical formulations, whether gels or liquids, with polymers. Embodiments include formulations including one or more suitable polymers. In certain embodiments the polymers can be selected from ethylene polymers, acrylic polymers, polyvinylpyrrolidones (PVPs), polyvinyl copolymers, cellulose polymers, including modified cellulose, natural polymers including collagen, polystyrene polymers, silicone polymers, inorganic polymers, and the like. Examples of ethylene polymers that can be used include, but are not limited to, oxidized polyethylene, polyethylene, polyethylene glycol, and the like. Examples of acrylic polymers that can be used include, but are not limited to, acrylic esters, methacrylic esters copolymer, acrylic polymer emulsion, carbomer, ethylene acrylates, methacryiol ethyl betaine, methacrylates copolymer, octylacrylamide, acrylates, butylaminoethyl methacrylate copolymer, polyacrylamidomethylpropane sulfonic acid, polyquaternium-5, polyquaternium-6, polyquaternium-7, polyquatemium-15, and the like. Examples of polyvinylpyrrolidones (PVPs) include, but are not limited to, polyquaternium-11, polyvinylpyrrolidone (PVP), PVP/dimethylaminoethylmethacrylate copolymers, PVP/Elcosene copolymer, PVP/ethyl methacrylate/methacrylic acid terpolymer, PVP/hexadecene copolymer, PVPNA copolymers, styrene/PVP copolymer, and the like. Examples of polyvinyl copolymers include, but are not limited to, ethylene vinyl acetate copolymer, PVM/MA copolymer esters, vinyl acetate/crotonic acid copolymer, vinyl acetate/crotonic acid/methacryloxybenzophenone-1 copolymer, vinyl acetate/cotonic acid/vinyl neodecanoate copolymer, carboxymethyl cellulose, hydroxyethyl cellulose, hydroxypropyl cellulose, hydroxypropyl methylcellulose, PEG celluloses, polyquaternium-4, polyquaternium-10, and the like. Examples of natural polymers include, but are not limited to, acacia, agar, alginate, carrageenan, furcelleran, gelatin, ghatti gum, glycosaminoglycans, guar gum, guar gum derivative, hydroxypropyl guar, hyaluronic acid, karaya, locust bean gum, maltodextrin, pectin, tragacanth gum, xanthan, and the like. Examples of polystyrene polymers include, but are not limited to, sodium polystyrene sulfonate. Examples of silicone polymers include amino bispropyl dimethicone, cyclomethicone, dimethicone, dimethicone copolyol, hexamethyldisiloxane, methicone, octadecyl dimethicone, phenyl dimethicone, stearoxy dimethicone, and the like. Examples of inorganic polymers, include but are not limited to bentonite, modified bentonite, magnesium aluminum silicate, modified hectorite, sodium magnesium silicate, and the like.

Illustrative formulations include an emulsion that includes a targeted nitroxide agent, at least one fatty acid (which may be provided by including an appropriate oil such as a plant-derived oil (e.g, a vegetable oil, a legume oil, a fruit oil, etc.), a phospholipid, and a nonionic surfactant. A specific formulation is a mixture of sesame oil:egg-Phosphatidyl Choline (PC):Tween80:Span85 at 1.0:0.5:0.25:0.25 w/w., and a targeted nitroxide agent (e.g., JP4-039) present in an amount of 2 mg/ml in this emulsion.

### Targeted Nitroxide Agents

Examples of targeted nitroxide agents are disclosed in WO 2010/009405, WO 2010/009389 and WO 2010/009327, all of which are incorporated herein by reference in their entireties.

The effective mitochondrial concentration of mitochondria targeted-conjugated nitroxides against γ-irradiation could be increased up to 1,000 times (and their required tissues concentrations can be reduced 1,000 times from 10 mM to 10 μM) compared with parent non-conjugated nitroxides. Enrichment in mitochondria of mitochondria targeted nitroxides has been demonstrated by EPR spectroscopy as well as by MS analysis of their content in mitochondria obtained from cells incubated with mitochondria targeted nitroxides. Delivery of mitochondria targeted-nitroxides into mitochondria does not depend on the mitochondrial membrane potential. Therefore, mitochondria targeted nitroxides can accumulate not only in intact but also in de-energized or damaged mitochondria with low membrane potential. Moreover, mitochondria targeted nitroxide conjugates are delivered into mitochondria without affecting the mitochondrial membrane potential, hence they do not impair the major mitochondrial function, the energy production, in cells. In addition, the conjugated nitroxides provide a new important feature, post irradiation protection.

Like other nitroxides, conjugated mitochondria targeted nitroxides might potentially lower blood pressure and sympathetic nerve activity. However, the dramatically reduced dose of mitochondria targeted nitroxides (about 1,000-fold), compared to non-conjugated parental nitroxides, may be significantly below of those inducing side effects.

An antioxidant compound is defined herein as a compound that decreases the rate of oxidation of other compounds or prevents a substance from reacting with oxygen or oxygen containing compounds. A compound may be determined to be an antioxidant compound by assessing its ability to decrease molecular oxidation and/or cellular sequellae of oxidative stress, for example, and without limitation, the ability to decrease lipid peroxidation and/or decrease oxidative damage to protein or nucleic acid. In one embodiment, an antioxidant has a level of antioxidant activity between 0.01 and 1000 times the antioxidant activity of ascorbic acid in at least one assay that measures antioxidant activity.

Provided herein are compounds and compositions comprising a targeting group and a nitroxide-containing group. The cargo may be any useful compound, such as an antioxidant, as are well known in the medical and chemical arts. The cargo may comprise a factor having anti-microbial activity. For example, the targeting groups may be cross-linked to antibacterial enzymes, such as lysozyme, or antibiotics, such as penicillin. Other methods for attaching the targeting groups to a cargo are well known in the art. In one embodiment, the cargo is an antioxidant, such as a nitroxide-containing group. In another embodiment, the cargo transported by mitochondria-selective targeting agents may include an inhibitor of NOS activity. The cargo may have a property selected from the group consisting of antioxidant, radioprotective, protective, anti-apoptotic, therapeutic, ameliorative, NOS antagonist and combinations thereof. In another embodiment, the cargo may have the ability to inhibit nitric oxide synthase enzyme activity. It will be appreciated that a wide variety of cargos may be employed in the composition described herein. Non-limiting examples of cargos include: a 2-amino-6-methyl-thiazine, a ubiquinone analog, a ubiquinone analog fragment moiety, a ubiquinone analog fragment moiety lacking a hydrophilic tail, a superoxide dismutase mimetic, a superoxide dismutase biomimetic and a salen-manganese compound.

While the generation of ROS in small amounts is a typical byproduct of the cellular respiration pathway, certain conditions, including a disease or other medical condition, may occur in the patient when the amount of ROS is excessive to the point where natural enzyme mechanisms cannot scavenge the amount of ROS being produced. Therefore, compounds, compositions and methods that scavenge reactive oxygen species that are present within the mitochondrial membrane of the cell are useful and are provided herein. These compounds, compositions and methods have the utility of being able to scavenge an excess amount of ROS being produced that naturally occurring enzymes SOD and catalase, among others, cannot cope with.

In one non-limiting embodiment, the compound has the structure:

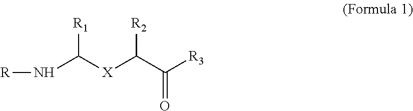

wherein X is one of

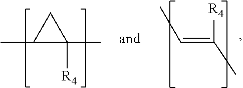

and
R₁, R₂ and R₄ are, independently, hydrogen, C₁-C₆ straight or branched-chain alkyl, optionally including a phenyl (C₆H₅) group, that optionally is methyl-, hydroxyl- or fluoro-substituted, including: methyl, ethyl, propyl, 2-propyl, butyl, t-butyl, pentyl, hexyl, benzyl, hydroxybenzyl (e.g., 4-hydroxybenzyl), phenyl and hydroxyphenyl. R₃ is —NH—R₅, —O—R₅ or —CH₂—R₅, where R₅ is an —N—O., —N—OH or N═O containing group. In one embodiment, R₃ is

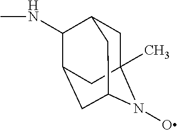

(1-Me-AZADO or 1-methyl azaadamantane N-oxyl). In another embodiment, R₃ is

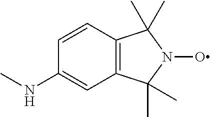

(TMIO; 1,1,3,3-tetramethylisoindolin-2-yloxyl).
R is —C(O)—R₆, —C(O)O—R₆, or —P(O)—(R₆)₂, wherein R₆ is C₁-C₆ straight or branched-chain alkyl optionally comprising one or more phenyl (—C₆H₅) groups, and that optionally are methyl-, ethyl-, hydroxyl- or fluoro-substituted, including Ac (Acetyl, R═—C(O)—CH₃), Boc (R═C(O)O-tert-butyl), Cbz (R═—C(O)O-benzyl (Bn)) groups. R also may be a diphenylphosphate group, that is,

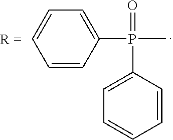

In certain embodiments, R₁ is t-butyl and R₂ and R₄ are H, for instance:

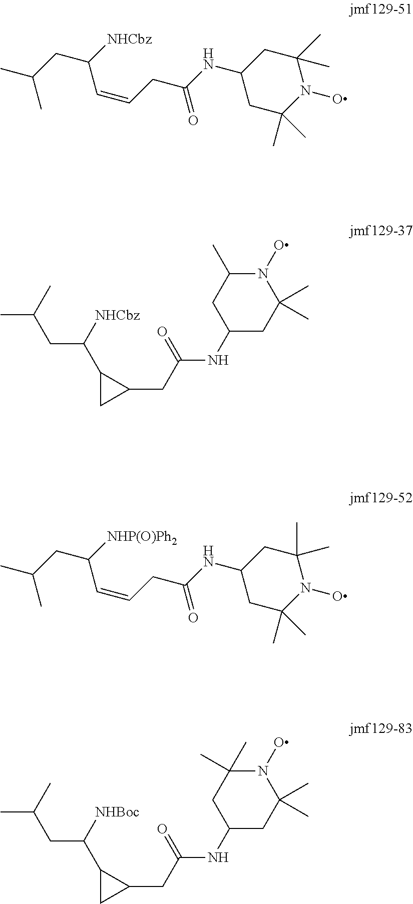

As used herein, unless indicated otherwise, for instance in a structure, all compounds and/or structures described herein comprise all possible stereoisomers, individually or mixtures thereof.

As indicated above, R₅ is an —N—O., —N—OH or —N═O containing group (not —N—O., —N—OH or —N═O, but groups containing those moieties, such as TEMPO, etc, as described herein). As is known to one ordinarily skilled in the art, nitroxide and nitroxide derivatives, including TEMPOL and associated TEMPO derivatives are stable radicals that can withstand biological environments. Therefore, the presence of the 4-amino-TEMPO, TEMPOL or another nitroxide “payload” within the mitochondria membrane can serve as an effective and efficient electron scavenger of the ROS being produced within the membrane. Non-limiting examples of this include TEMPO (2,2,6,6-Tetramethyl-4-piperidine 1-oxyl) and TEMPOL (4-Hydroxy-TEMPO), in which, when incorporated into the compound described herein, form, for example, when R₃ is —NH—R₅, —O—R₅:

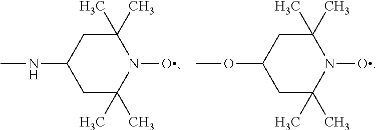

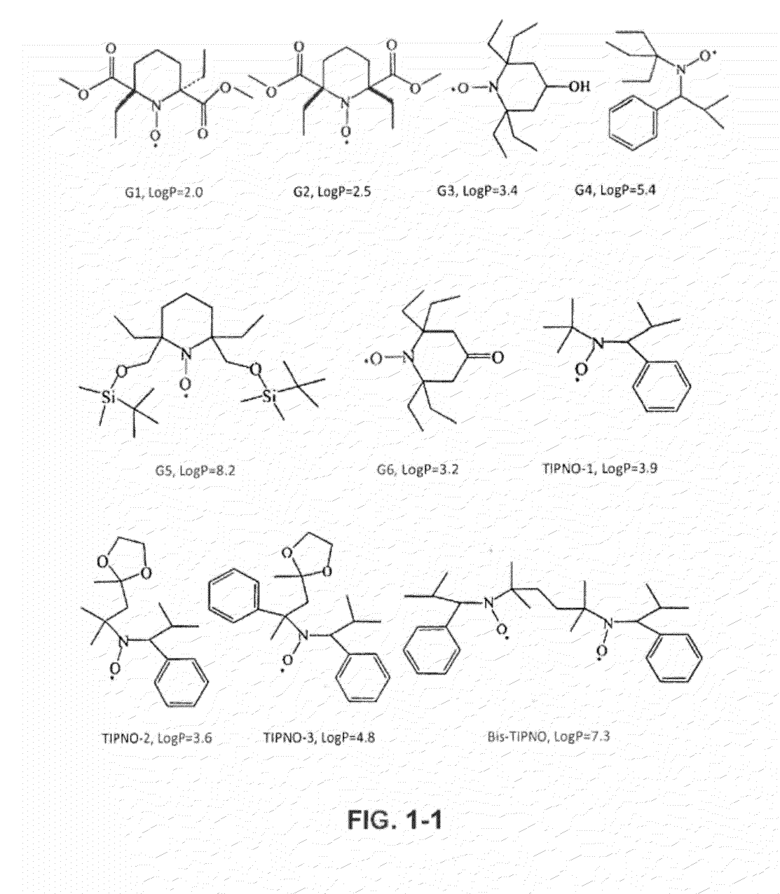

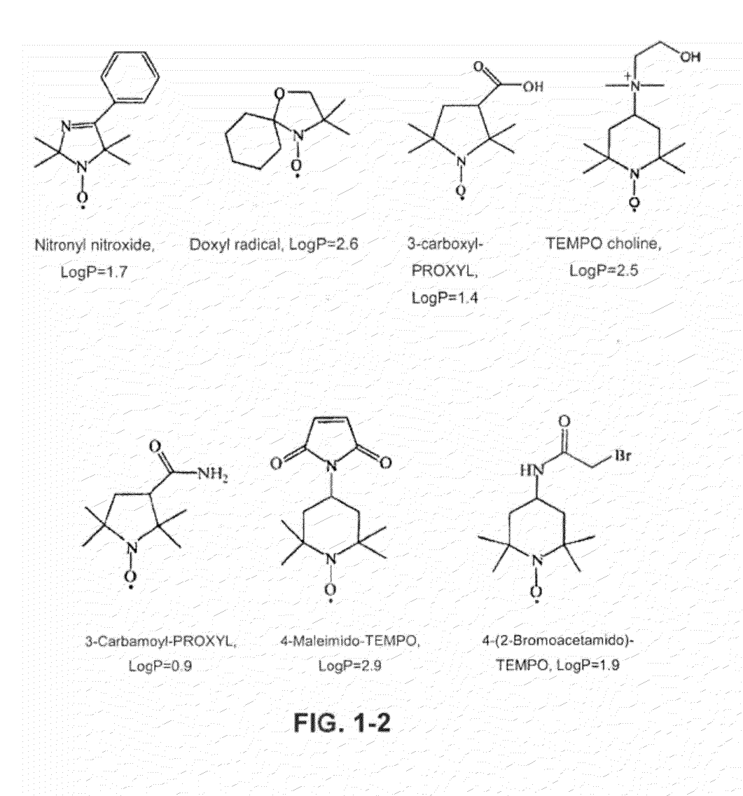

Additional non-limiting examples of —N—O., —N—OH or N═O containing group are provided in Table 1 and in FIG. 1 (from Jiang, J., et al. “Structural Requirements for Optimized Delivery, Inhibition of Oxidative Stress, and Antiapoptotic Activity of Targeted Nitroxides”, J Pharmacol Exp Therap. 2007, 320(3):1050-60). A person of ordinary skill in the art would be able to conjugate (covalently attach) any of these compounds to the rest of the compound using common linkers and/or conjugation chemistries, such as the chemistries described herein. Table 1 provides a non-limiting excerpt from a list of over 300 identified commercially-available —N—O., —N—OH or N═O containing compounds that may be useful in preparation of the compounds or compositions described herein.

TABLE 1
Commercially-available —N—O•, —N—OH or N═O containing groups

| Structure | Name | CAS No. |
|---|---|---|
| 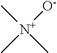 | Trimethylamine N- Oxide | 1184-78-7 |
| 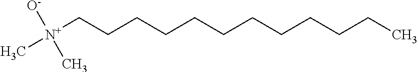 | N,N- Dimethyldodecylamine N-Oxide | 1643-20-5 70592-80-2 |
| 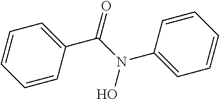 | N-Benzoyl-N- Phenylhydroxylamine | 304-88-1 |
| 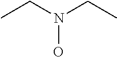 | N,N- Diethylhydroxylamine | 3710-84-7 |
| 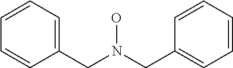 | N,N- Dibenzylhydroxylamine | 14165-27-6 621-07-8 |
| 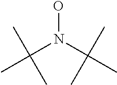 | Di-Tert-Butyl Nitroxide | 2406-25-9 |
| 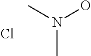 | N,N- Dimethylhydroxylamine Hydrochloride | 16645-06-0 |
| 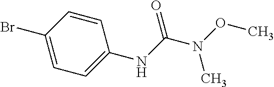 | Metobromuron | 3060-89-7 |
| 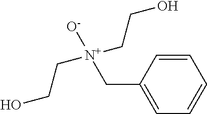 | Benzyl-Di-Beta- Hydroxy Ethylamine-N- Oxide | |
| 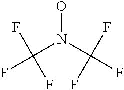 | Bis(Trifluoromethyl) Nitroxide | 2154-71-4 |
| 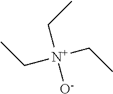 | Triethylamine N- Oxide | 2687-45-8 |

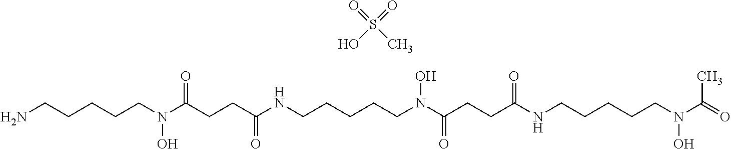

| | | |
|---|---|---|
| 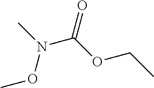 | N-Methoxy-N- Methylcarbamate | 6919-62-6 |
| 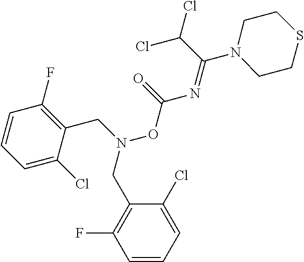 | N,N-BIS(2- CHLORO-6- FLUOROBENZYL)- N-[(([2,2- DICHLORO-1-(1,4- THIAZINAN-4- YL+) ETHYLIDENE] AMINO)CARBONYL) OXY]AMINE | |
| 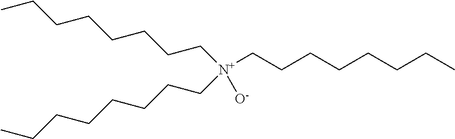 | Tri-N-Octylamine N-Oxide | 13103-04-3 |
| 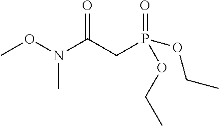 | DIETHYL (N- METHOXY-N- METHYLCARBAMOYLMETHYL) PHOSPHONATE | 124931-12-0 |
| 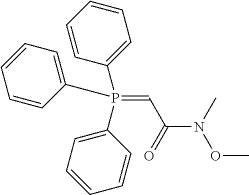 | N-Methoxy-N- Methyl-2- (Triphenylphosphoranylidene) Acetamide | 129986-67-0 |
| 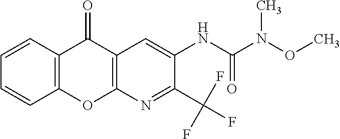 | N-Methoxy-N- Methyl-N′-[5-Oxo- 2-(Trifluoromethyl)- 5h-Chromeno[2,3- B]Pyridi+ N-3-Yl]Urea | |
| 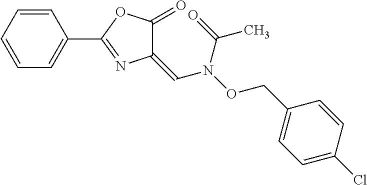 | N-[(4- Chlorobenzyl)Oxy]- N-([5-Oxo-2- Phenyl-1,3-Oxazol- 4(5h)- Yliden]Methyl+) Acetamide | |
| 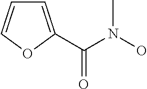 | N- Methylfurohydroxamic Acid | 109531-96-6 |
| 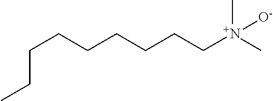 | N,N- Dimethylnonylamine N-Oxide | 2536-13-2 |
| 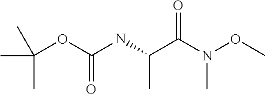 | N-(Tert- Butoxycarbonyl)-L- Alanine N′- Methoxy-N′- Methylamide | 87694-49-3 |
| 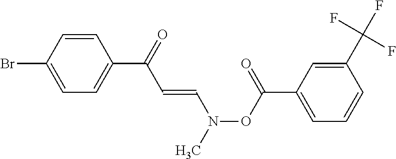 | 1-(4-Bromophenyl)- 3-(Methyl([3- (Trifluoromethyl) Benzoyl]Oxy)Amino)- 2-Prop+ En-1-One | |
| 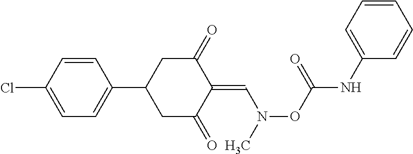 | 2- ([[(Anilinocarbonyl) Oxy](Methyl)Amino] Methylene)-5-(4- Chlorophenyl)-1,3+- Cyclohexanedione | |
| 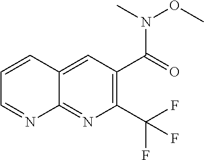 | N-Methoxy-N- Methyl-2- (Trifluoromethyl)- 1,8-Naphthyridine- 3-Carboxamide | |
| 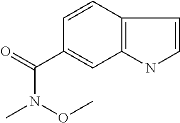 | N-Methoxy-N- Methyl-Indole-6- Carboxamide | |

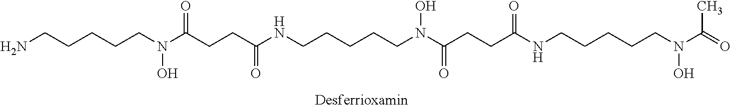

| | | |
|---|---|---|
| 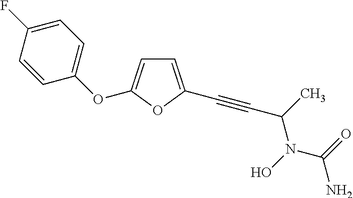 | AKOS 91254 | 127408-31-5 |
| 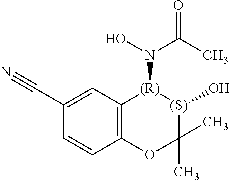 | N-[(3s,4r)-6-Cyano- 3,4-Dihydro-3- Hydroxy-2,2- Dimethyl-2h-1- Benzopyran-4-Y+ L]-N- Hydroxyacetamide | 127408-31-5 |
| 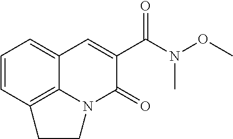 | N-Methoxy-N- Methyl-1,2- Dihydro-4-Oxo- Pyrrolo[3,2,1- Ij]Quinoline-5- Carboxa+ Mide | |
| 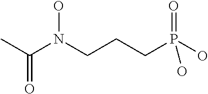 | Fr-900098 | |
| 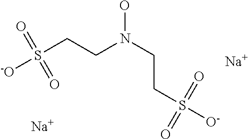 | 2,2′- (Hydroxyimino)Bis- Ethanesulfonic Acid Disodium Salt | 133986-51-3 |
| 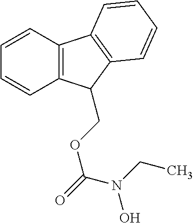 | Fmoc-N-Ethyl- Hydroxylamine | |
| 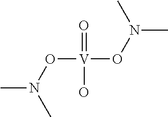 | Bis(N,N- Dimethylhydroxamido) Hydroxooxovanadate | |
| 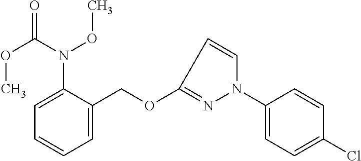 | Pyraclostrobin | 175013-18-0 |
| 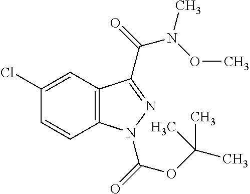 | 1-Boc-5-Chloro-3- (Methoxy-Methyl- Carbamoyl)Indazole | |
| 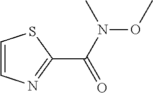 | N-Methoxy-N- Methyl-Thiazole-2- Carboxamide | |
| 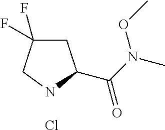 | 4,4-Difluoro-N- Methyl-N-Methoxy- L-Prolinamide Hcl | |
| 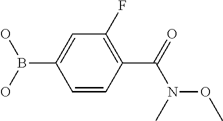 | 3-Fluoro-4- (Methoxy(Methyl) Carbamoyl)Phenylboronic Acid | 913835-59-3 |
| 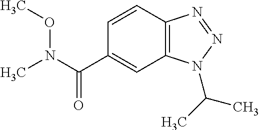 | 1-Isopropyl-N- Methoxy-N-Methyl- 1h- Benzo[D][1,2,3] Triazole-6-Carboxamide | 467235-06-9 |
| 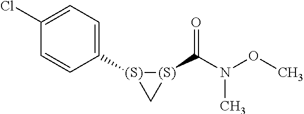 | (Trans)-2-(4- Chlorophenyl)-N- Methoxy-N- Methylcyclopropane carboxamide | |
| 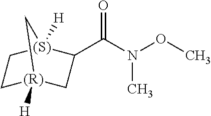 | Bicyclo[2.2.1]Heptane- 2-Carboxylic Acid Methoxy- Methyl-Amide | |
| 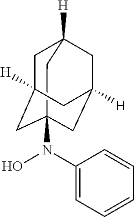 | Akos Bc-0582 | |
| 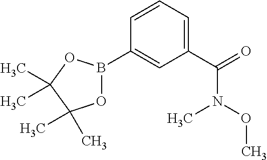 | 3-(N,O- Dimethylhydroxylaminocarbonyl) Phenylboronic Acid, Pinacol Ester | |
| 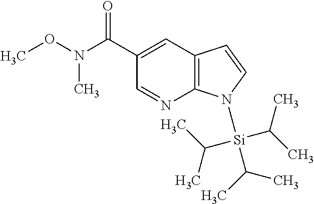 | 1- Triisopropylsilanyl- 1h-Pyrrolo[2,3- B]Pyridine-5- Carboxylic Acid Methoxy+- Methyl-Amide | |

According to one embodiment, the compound has the structure

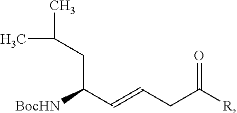

or the structure

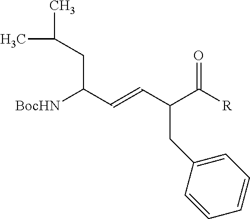

wherein R is —NH—R₁, —O—R₁ or —CH₂—R₁, and R₁ is an —N—O., —N—OH or N═O containing group. In one embodiment, R is —NH—R₁, and in another R is —NH-TEMPO.

According to another embodiment, the compound has the structure:

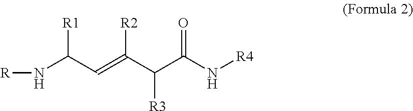

in which R1, R2 and R3 are, independently, hydrogen, C₁-C₆ straight or branched-chain alkyl, optionally including a phenyl (C₆H₅) group, that optionally is methyl-, hydroxyl- or fluoro-substituted, including 2-methyl propyl, benzyl, methyl-, hydroxyl- or fluoro-substituted benzyl, such as 4-hydroxybenzyl. R4 is an —N—O., —N—OH or N═O containing group. In one embodiment, R4 is

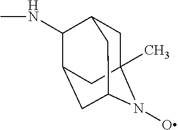

(1-Me-AZADO or 1-methyl azaadamantane N-oxyl). In another embodiment R4 is

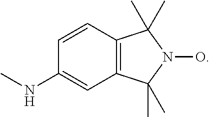

(TMIO; 1,1,3,3-tetramethylisoindolin-2-yloxyl). R is —C(O)—R5, —C(O)O—R5, or —P(O)—(R5)₂, wherein R5 is C₁-C₆ straight or branched-chain alkyl, optionally comprising one or more phenyl (—C₆H₅) groups, and that optionally are methyl-, ethyl-, hydroxyl- or fluoro-substituted, including Ac, Boc, and Cbz groups. R also may be a diphenylphosphate group, that is,

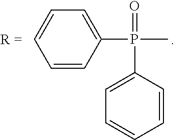

In certain specific embodiments, in which R4 is TEMPO, the compound has one of the structures A, A1, A2, or A3 (Ac=Acetyl=CH₃C(O)—):

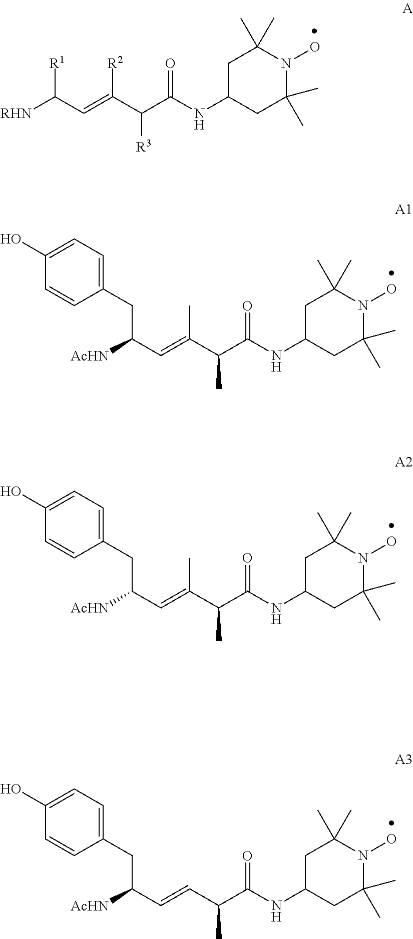

According to another embodiment, the compound has the structure

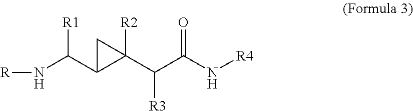

In which R1, R2 and R3 are, independently, hydrogen, C₁-C₆ straight or branched-chain alkyl, optionally including a phenyl (C₆H₅) group, that optionally is methyl-, hydroxyl- or fluoro-substituted, including 2-methyl propyl, benzyl, methyl-, hydroxyl- or fluoro-substituted benzyl, such as 4-hydroxybenzyl. R4 is an —N—O., —N—OH or N═O containing group. In one embodiment, R4 is

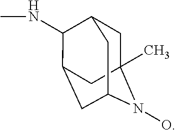

(1-Me-AZADO or 1-methyl azaadamantane N-oxyl). In another embodiment R4 is

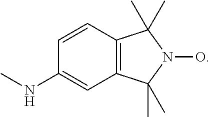

(TMIO; 1,1,3,3-tetramethylisoindolin-2-yloxyl). R is —C(O)—R5, —C(O)O—R5, or —P(O)—(R5)₂, wherein R5 is C₁-C₆ straight or branched-chain alkyl, optionally comprising one or more phenyl (—C₆H₅) groups, and that optionally are methyl-, ethyl-, hydroxyl- or fluoro-substituted, including Ac, Boc, and Cbz groups. R also may be a diphenylphosphate group, that is,

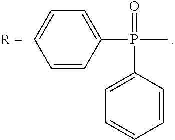

In certain specific embodiments, in which R4 is TEMPO, the compound has one of the structures D, D1, D2, or D3 (Ac=Acetyl=CH₃C(O)—):

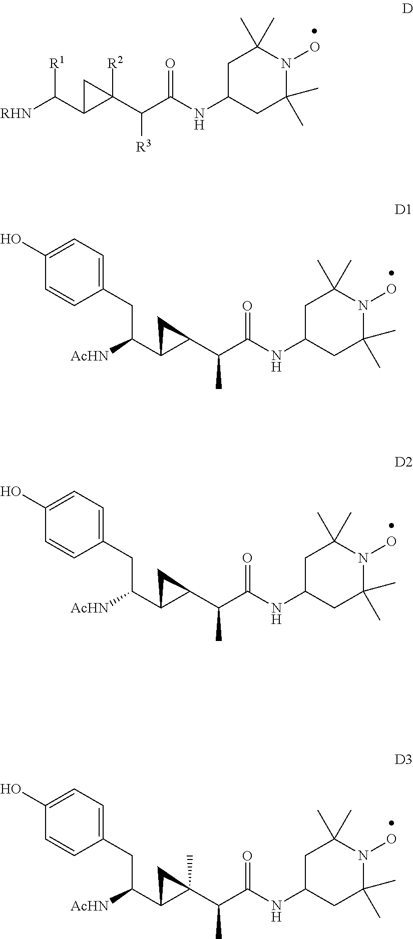

In another non-limiting embodiment, the compound has the structure:

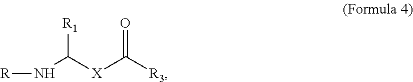

wherein X is one of

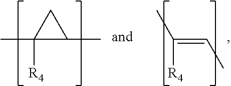

and
R₁ and R₄ are, independently, hydrogen, C₁-C₆ straight or branched-chain alkyl, optionally including a phenyl (C₆H₅) group, that optionally is methyl-, hydroxyl- or fluoro-substituted, including: methyl, ethyl, propyl, 2-propyl, butyl, t-butyl, pentyl, hexyl, benzyl, hydroxybenzyl (e.g., 4-hydroxybenzyl), phenyl and hydroxyphenyl. R₃ is —NH—R₅, —O—R₅ or —CH₂—R₅, where R₅ is an —N—O., —N—OH or N═O containing group. In one embodiment, R₃ is

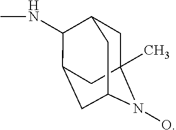

(1-Me-AZADO or 1-methyl azaadamantane N-oxyl). In another embodiment, R₃ is

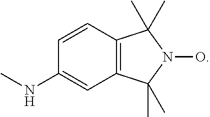

(TMIO; 1,1,3,3-tetramethylisoindolin-2-yloxyl).
R is —C(O)—R₆, —C(O)O—R₆, or —P(O)—(R₆)₂, wherein R₆ is C₁-C₆ straight or branched-chain alkyl optionally comprising one or more phenyl (—C₆H₅) groups, and that optionally are methyl-, ethyl-, hydroxyl- or fluoro-substituted, including Ac (Acetyl, R═—C(O)—CH₃), Boc (R═—C(O)O-tert-butyl), Cbz (R═—C(O)O-benzyl (Bn)) groups. R also may be a diphenylphosphate group, that is,

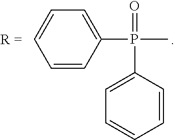

In one non-limiting embodiment, the compound has one of the structures

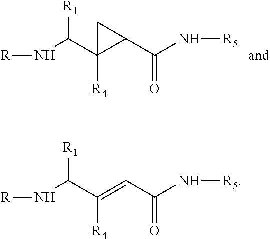

In yet another non-limiting embodiment, the compound has the structure

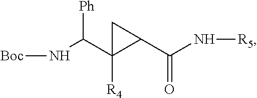

in which R₄ is hydrogen or methyl.

The compounds described above, such as the compound of Formula I, can be synthesized by any useful method. The compound JP4-039 was synthesized by the method of Example 5. In one embodiment, a method of making a compound of formula I is provided. The compounds are synthesized by the following steps:

A. reacting an aldehyde of structure R₁—C(O)—, wherein, for example and without limitation, R₁ is C₁-C₆ straight or branched-chain alkyl, optionally including a phenyl (C₆H₅) group, that optionally is methyl-, hydroxyl- or fluoro-substituted, including: methyl, ethyl, propyl, 2-propyl, butyl, t-butyl, pentyl, hexyl, benzyl, hydroxybenzyl (e.g., 4-hydroxybenzyl), phenyl and hydroxyphenyl, with (R)-2-methylpropane-2-sulfinamide to form an imine, for example

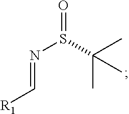

B. reacting a terminal alkyne-1-ol (CHC—R₂—C—OH), wherein, for example and without limitation, R₂ is not present or is branched or straight-chained alkylene, including methyl, ethyl, propyl, etc., with a tert-butyl)diphenylsilane salt to produce an alkyne, for example

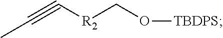

C. reacting (by hydrozirconation) the alkyne with the imine in the presence of an organozirconium catalyst to produce an alkene, for example

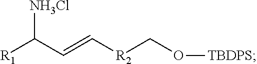

D1. acylating the alkene to produce a carbamate, for example

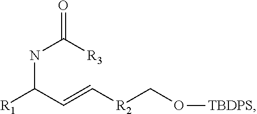

wherein, for example and without limitation, R₃ is C₁-C₆ straight or branched-chain alkyl, optionally including a phenyl (C₆H₅) group, that optionally is methyl-, hydroxyl- or fluoro-substituted, including: methyl, ethyl, propyl, 2-propyl, butyl, t-butyl, pentyl, hexyl, benzyl, hydroxybenzyl (e.g., 4-hydroxybenzyl), phenyl and hydroxyphenyl;
D2. optionally, cyclopropanating the alkene and then acylating the alkene to produce a carbamate, for example

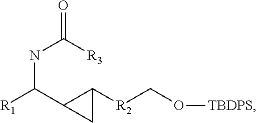

wherein, for example and without limitation, R₃ is C₁-C₆ straight or branched-chain alkyl, optionally including a phenyl (C₆H₅) group, that optionally is methyl-, hydroxyl- or fluoro-substituted, including: methyl, ethyl, propyl, 2-propyl, butyl, t-butyl, pentyl, hexyl, benzyl, hydroxybenzyl (e.g., 4-hydroxybenzyl), phenyl and hydroxyphenyl;
E. removing the t-butyldiphenylsilyl group from the carbamate to produce an alcohol, for example

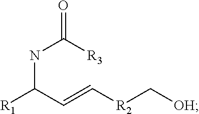

F. oxidizing the alcohol to produce a carboxylic acid, for example

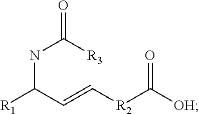

and
G. reacting the carboxylic acid with a nitroxide-containing compound comprising one of a hydroxyl or amine in a condensation reaction to produce the antioxidant compound, for example

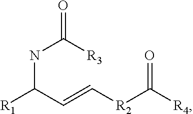

wherein R₄ is —NH—R₄ or —O—R₄, and R₄ is an —N—O., —N—OH or N═O containing group, such as described above.

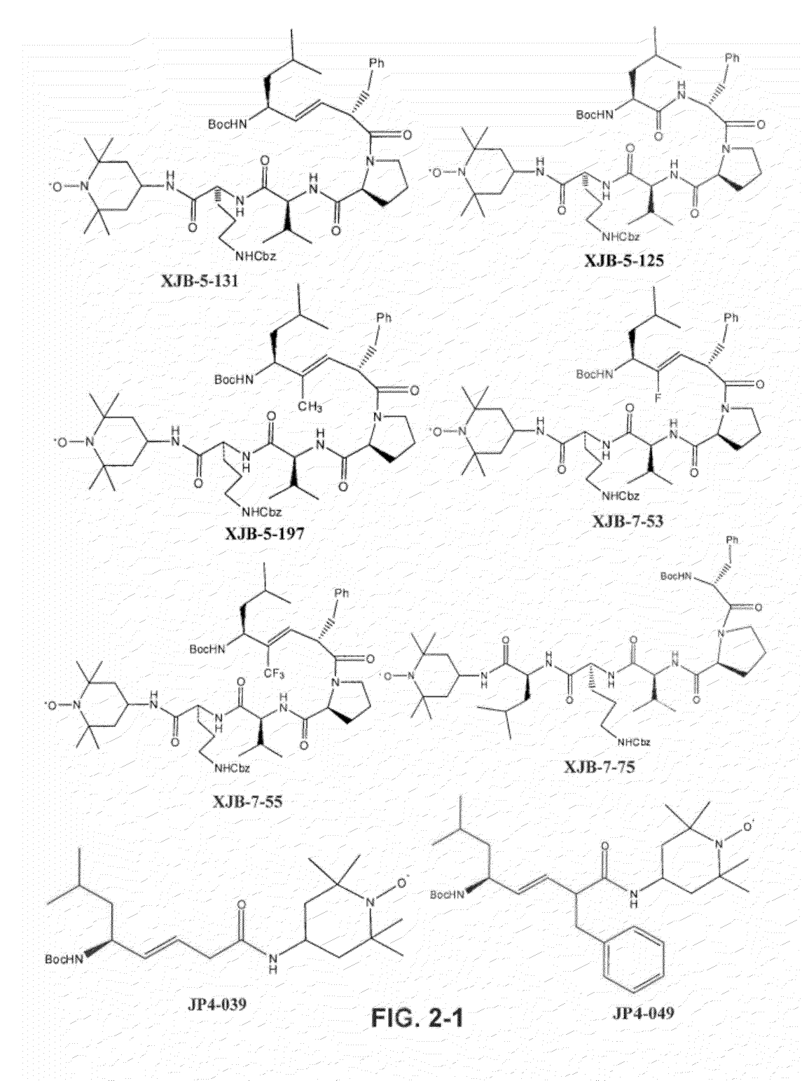

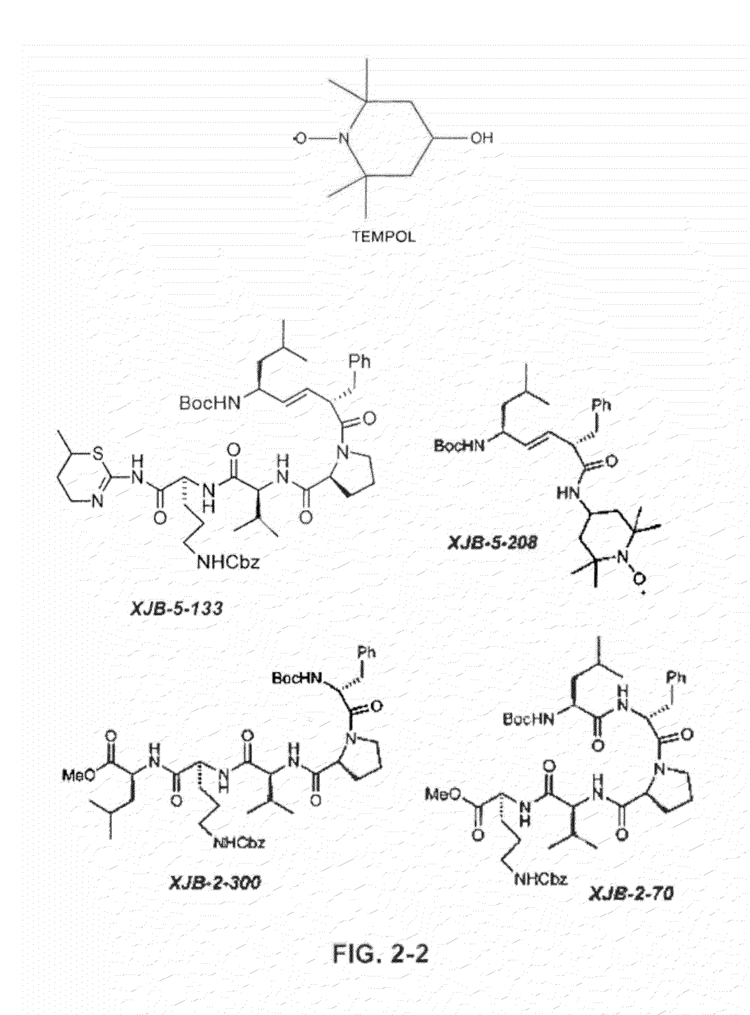

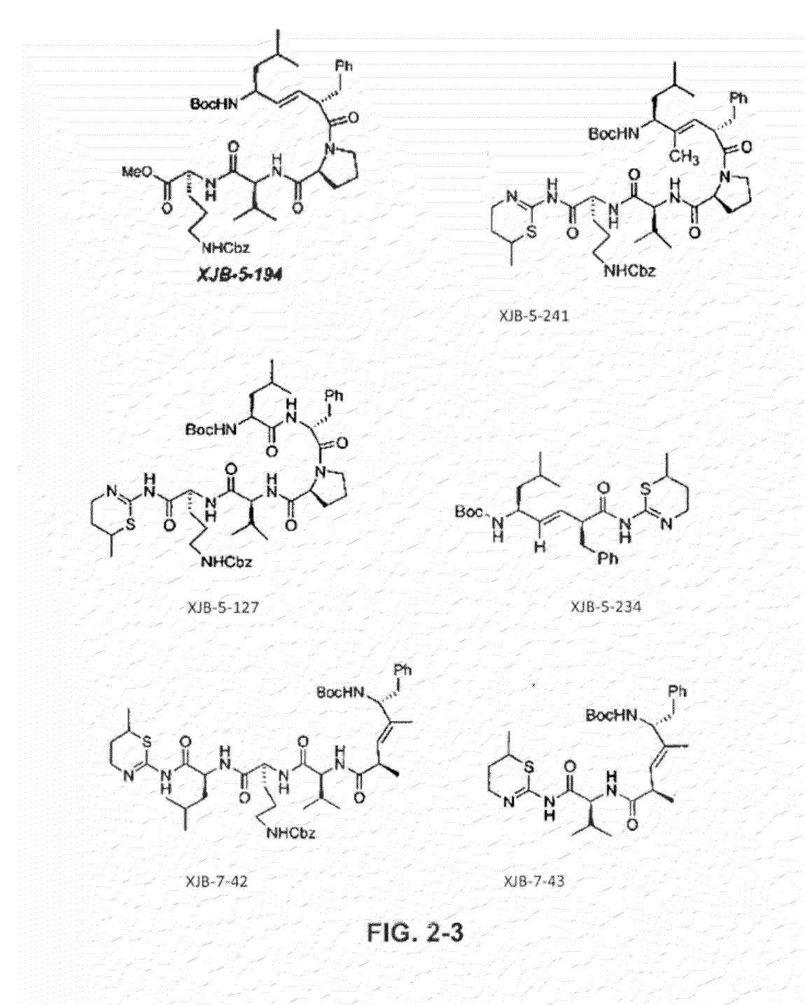

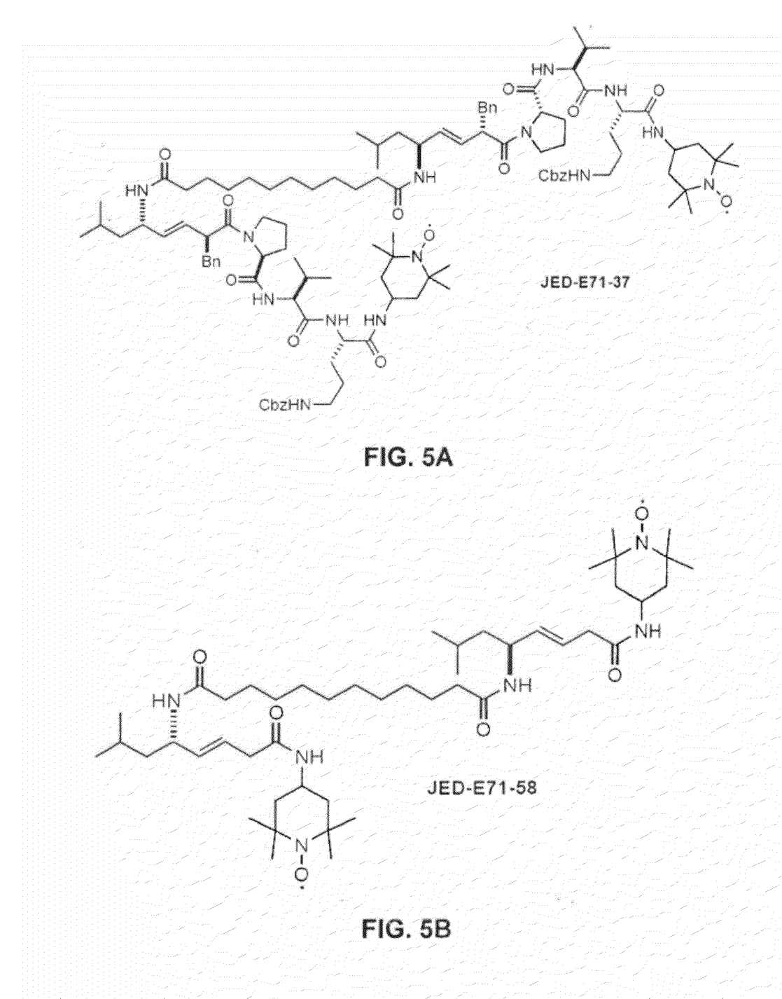

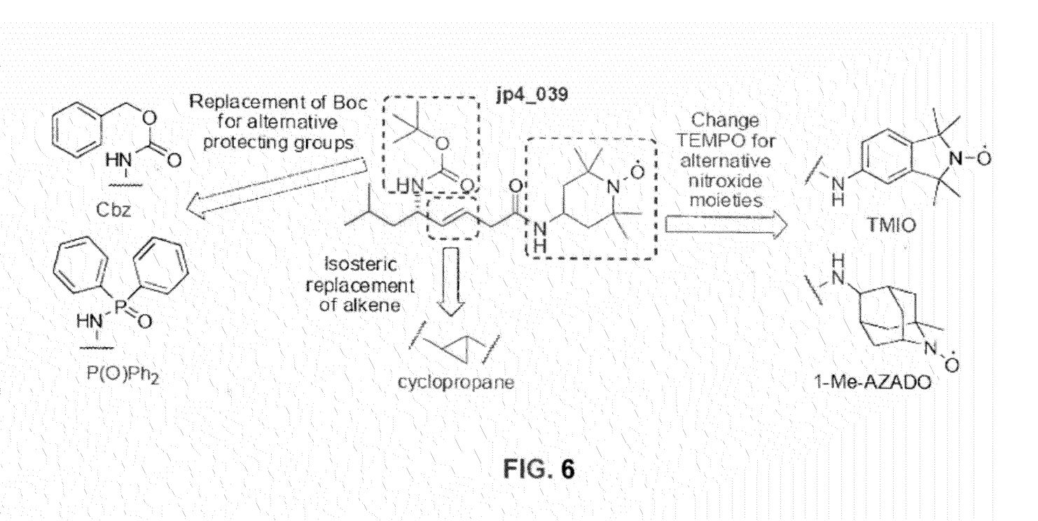

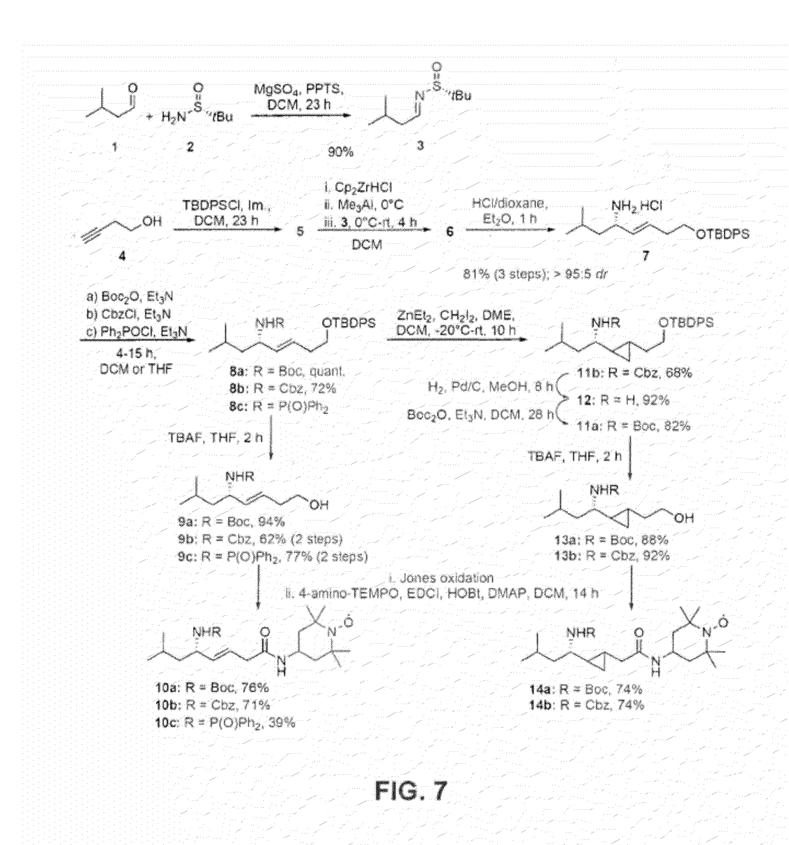

In another non-limiting embodiment, a compound is provided having the structure R1-R2-R3 in which R1 and R3 are a group having the structure —R4-R5, in which R4 is a mitochondria targeting group and R5 is —NH—R6, —O—R6 or —CH₂—R6, wherein R6 is an —N—O., —N—OH or N═O containing group, such as TEMPO. R1 and R2 may be the same or different. Likewise, R4 and R5 for each of R1 and R3 may be the same or different. R2 is a linker that, in one non-limiting embodiment, is symmetrical. FIGS. 5A and 5B depicts two examples of such compounds. In one embodiment, R1 and R2 have the structure shown in formulas 1, 2, or 3, above, with all groups as defined above, including structures A, A1, A2 A3, D, D1, D2 and D3, above, an example of which is compound JED-E71-58, shown in FIG. 5B. In another embodiment, R1 and R2 are, independently, a gramicidin derivative, for example, as in the compound JED-E71-37, shown in FIG. 5A. Examples of gramicidin derivatives are provided herein, such as XJB-5-131 and XJB-5-125 (see, FIG. 2), and are further described both structurally and functionally in United States Patent Publication Nos. 20070161573 and 20070161544 as well as in Jiang, J, et al. (Structural Requirements for Optimized Delivery, Inhibition of Oxidative Stress, and Antiapoptotic Activity of Targeted Nitroxides, J Pharmacel Exp Therap. 2007, 320(3):1050-60, see also, Hoye, A T et al., Targeting Mitochondria, Acc Chem. Rea. 2008, 41(1):87-97, see also, Wipf, P, et al., Mitochondrial Targeting of Selective Electron Scavengers Synthesis and Biological Analysis of Hemigramicidin-TEMPO Conjugates, J Am Chem. Soc. 2005, 127, 12460-12461). The XJB compounds can be linked into a dimer, for example and without limitation, by reaction with the nitrogen of the BocHN groups (e.g., as in XJB-5-131), or with an amine, if present, for instance, if one or more amine groups of the compound is not acylated to form an amide (such as NHBoc or NHCbx).

In Jiang, J, et al. (J Pharmacol Exp Therap. 2007, 320(3):1050-60), using a model of ActD-induced apoptosis in mouse embryonic cells, the authors screened a library of nitroxides to explore structure-activity relationships between their antioxidant/antiapoptotic properties and chemical composition and three-dimensional (3D) structure. High hydrophobicity and effective mitochondrial integration were deemed necessary but not sufficient for high antiapoptotic/antioxidant activity of a nitroxide conjugate. By designing conformationally preorganized peptidyl nitroxide conjugates and characterizing their 3D structure experimentally (circular dichroism and NMR) and theoretically (molecular dynamics), they established that the presence of the β-turn/β-sheet secondary structure is essential for the desired activity. Monte Carlo simulations in model lipid membranes confirmed that the conservation of the D-Phe-Pro reverse turn in hemi-GS analogs ensures the specific positioning of the nitroxide moiety at the mitochondrial membrane interface and maximizes their protective effects. These insights into the structure-activity relationships of nitroxide-peptide and -peptide isostere conjugates are helpful in the development of new mechanism-based therapeutically effective agents, such as those described herein.

Targeting group R4 may be a membrane active peptide fragment derived from an antibiotic molecule that acts by targeting the bacterial cell wall. Examples of such antibiotics include: bacitracins, gramicidins, valinomycins, enniatins, alamethicins, beauvericin, serratomolide, sporidesmolide, tyrocidins, polymyxins, monamycins, and lissoclinum peptides. The membrane-active peptide fragment derived from an antibiotic may include the complete antibiotic polypeptide, or portions thereof having membrane, and preferably mitochondria-targeting abilities, which is readily determined, for example, by cellular partitioning experiments using radiolabeled peptides. Examples of useful gramicidin-derived membrane active peptide fragments are the Leu-D-Phe-Pro-Val-Orn and D-Phe-Pro-Val-Orn-Leu hemigramicidin fragments. As gramicidin is cyclic, any hemigramicidin 5-mer is expected to be useful as a membrane active peptide fragment, including Leu-D-Phe-Pro-Val-Orn, D-Phe-Pro-Val-Orn-Leu, Pro-Val-Orn-Leu-D-Phe, Val-Orn-Leu-D-Phe-Pro and Om-Leu-D-Phe-Pro-Val (from Gramicidin S). Any larger or smaller fragment of gramicidin, or even larger fragments containing repeated gramicidin sequences (e.g., Leu-D-Phe-Pro-Val-Orn-Leu-D-Phe-Pro-Val-Orn-Leu-D-Phe-Pro) are expected to be useful for membrane targeting, and can readily tested for such activity. In one embodiment, the Gramicidin S-derived peptide comprises a β-turn, which appears to confer to the peptide a high affinity for mitochondria. Derivatives of Gramicidin, or other antibiotic fragments, include isosteres (molecules or ions with the same number of atoms and the same number of valence electrons—as a result, they can exhibit similar pharmacokinetic and pharmacodynamic properties), such as (E)-alkene isosteres (see, United States Patent Publication Nos. 20070161573 and 20070161544 for exemplary synthesis methods). As with Gramicidin, the structure (amino acid sequence) of bacitracins, other gramicidins, valinomycins, enniatins, alamethicins, beauvericin, serratomolide, sporidesmolide, tyrocidins, polymyxins, monamycins, and lissoclinum peptides are all known, and fragments of these can be readily prepared and their membrane-targeting abilities can easily be confirmed by a person of ordinary skill in the art.

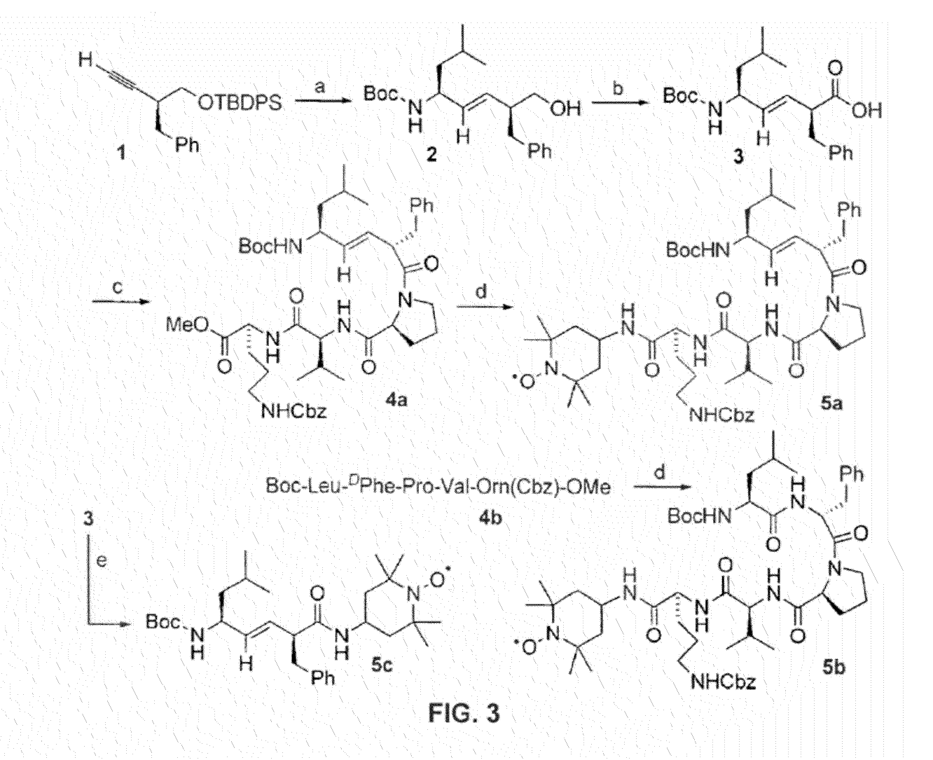

Alkene isosteres such as (E)-alkene isosteres of Gramicidin S (i.e., hemigramicidin) were used as part of the targeting sequence. See FIG. 3 for a synthetic pathway for (E)-alkene isosteres and reference number 2 for the corresponding chemical structure. First, hydrozirconation of alkyne (FIG. 3, compound 1) with Cp₂ZrHCl is followed by transmetalation to Me₂Zn and the addition of N-Boc-isovaleraldimine. The resulting compound (not shown) was then worked up using a solution of tetrabutylammonium fluoride (“TBAF”) and diethyl ether with a 74% yield. The resulting compound was then treated with acetic anhydride, triethylamine (TEA), and 4-N,N′-(dimethylamino) pyridine (“DMAP”) to provide a mixture of diastereomeric allylic amides with a 94% yield which was separated by chromatography. Finally, the product was worked up with K₂CO₃ in methanol to yield the (E)-alkene, depicted as compound 2. The (E)-alkene, depicted as compound 2 of FIG. 3, was then oxidized in a multi-step process to yield the compound 3 (FIG. 3)—an example of the (E)-alkene isostere.

The compound 3 of FIG. 3 was then conjugated with the peptide H-Pro-Val-Orn (Cbz)-OMe using 1-ethyl-3-(3-dimethylaminopropyl carbodiimide hydrochloride) (EDC) as a coupling agent. The peptide is an example of a suitable targeting sequence having affinity for the mitochondria of a cell. The resulting product is shown as compound 4a in FIG. 3. Saponification of compound 4a followed by coupling with 4-amino-TEMPO (4-AT) afforded the resulting conjugate shown as compound 5a in FIG. 3, in which the Leu-DPhe peptide bond has been replaced with an (E)-alkene.

In an alternate embodiment, conjugates 5b in FIG. 3 was prepared by saponification and coupling of the peptide 4b (Boc-Leu-^{D}Phe-Pro-Val-Orn(Cbz)-OMe) with 4-AT. Similarly, conjugate 5c in FIG. 3 was prepared by coupling the (E)-alkene isostere as indicated as compound 3 in FIG. 3 with 4-AT. These peptide and peptide analogs are additional examples of suitable targeting sequences having an affinity to the mitochondria of a cell.

In another embodiment, peptide isosteres may be employed as the conjugate. Among the suitable peptide isosteres are trisubstituted (E)-alkene peptide isosteres and cyclopropane peptide isosteres, as well as all imine addition products of hydro- or carbometalated internal and terminal alkynes for the synthesis of di and trisubstituted (E)-alkene and cyclopropane peptide isosteres. See Wipf et al. Imine additions of internal alkynes for the synthesis of trisubstituted (E)-alkene and cyclopropane isosteres, Adv Synth Catal. 2005, 347:1605-1613. These peptide mimetics have been found to act as β-turn promoters. See Wipf et al. Convergent Approach to (E)-Alkene and Cyclopropane Peptide Isosteres, Org. Lett. 2005, 7(1):103-106.

The linker, R2, may be any useful linker, chosen for its active groups, e.g., carboxyl, alkoxyl, amino, sulfhydryl, amide, etc. Typically, aside from the active groups, the remainder is non-reactive (such as saturated alkyl or phenyl), and does not interfere, sterically or by any other physical or chemical attribute, such as polarity or hydrophobicity/hydrophilicity, in a negative (loss of function) capacity with the activity of the overall compound. In one embodiment, aside from the active groups, the linker comprises a linear or branched saturated C₄-C₂₀ alkyl. In one embodiment, the linker, R2 has the structure

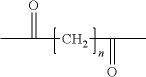

in which n is 4-18, including all integers therebetween, in one embodiment, 8-12, and in another embodiment, 10.

A person skilled in the organic synthesis arts can synthesize these compounds by crosslinking groups R1 and R3 by any of the many chemistries available. In one embodiment, R1 and R3 are to R2 by an amide linkage (peptide bond) formed by dehydration synthesis (condensation) of terminal carboxyl groups on the linker and an amine on R1 and R3 (or vice versa). In one embodiment, R1 and R3 are identical or different and are selected from the group consisting of: XJB-5-131, XJB-5-125, XJB-7-75, XJB-2-70, XJB-2-300, XJB-5-208, XJB-5-197, XJB-5-194, .JP4-039 and JP4-049, attached in the manner shown in FIGS. 5A and 5B.

According to another embodiment, the targeted nitroxide agent has a structure of:

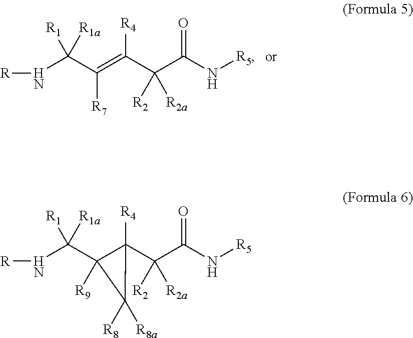

wherein R₁, R₁ₐ, R₂, and R₂ₐ are independently hydrogen, a halo, C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₄ is hydrogen, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or a C₁-C₆ straight or branched-chain alkyl further comprising one or more (C₆H₅) groups that are independently unsubstituted, or methyl-, ethyl-, hydroxyl- or halo-substituted; R₇, R₈, R₈ₐ, and R₉ are independently H, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted, provided that at least one of R₁, R₁ₐ, R₂, R₂ₐ, or R₇ is not H.

Substituted components can be singly or multiply substituted with the substituents set forth above. As use herein, halo refers to F, Cl, Br, or I. In a number of embodiments, components substituted with a halo atom are substituted with a fluorine atom (F).

In a number of embodiments of Formula 5 or Formula 6, R₂ and R₂ₐ are each F.

In certain embodiments of Formula 5 or Formula 6, R₁ is a C₁-C₆ straight or branched-chain alkyl (particularly tert-butyl); R₁ₐ is H; R₂ and R₂ₐ are each independently a halo (particularly F); R₇ is H or a C₁-C₆ straight or branched-chain alkyl; R₄ is H; R₅ is an —N—O.— containing group; and R is —C(O)O—R₆ wherein R₆ is C₁-C₆ straight or branched-chain alkyl (particularly tert-butyl). In certain embodiments, R₅ is

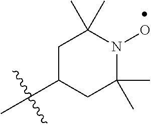

Further illustrative targeted nitroxide agents include:

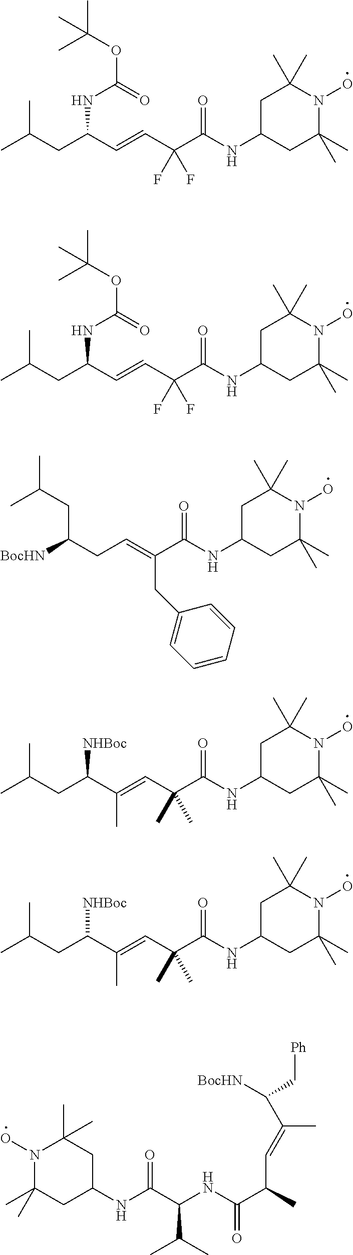

### General Radiation Protection

In any case, as used herein, any agent or agents used for prevention, mitigation or treatment in a subject of injury caused by radiation exposure is administered in an amount effective to prevent, mitigate of treat such injury, namely in an amount and in a dosage regimen effective to prevent injury or to reduce the duration and/or severity of the injury resulting from radiation exposure. According to one non-limiting embodiment, an effective dose ranges from 0.1 or 1 mg/kg to 100 mg/kg, including any increment or range therebetween, including 1 mg/kg, 5 mg/kg, 10 mg/kg, 20 mg/kg, 25 mg/kg, 50 mg/kg, and 75 mg/kg. However, for each compound described herein, an effective dose or dose range is expected to vary from that of other compounds described herein for any number of reasons, including the molecular weight of the compound, bioavailability, specific activity, etc. For example and without limitation, where XJB-5-131 is the antioxidant, the dose may be between about 0.1 and 20 mg/kg, or between about 0.3 and 10 mg/kg, or between about 2 and 8 mg/kg, or about 2 mg/kg and where either JP4-039, JED-E71-37 or JED-E71-58 is the antioxidant, the dose may be between about 0.01 and 50 mg/kg, or between about 0.1 and 20 mg/kg, or between about 0.3 and 10 mg/kg, or between about 2 and 8 mg/kg, or about 2 mg/kg. The therapeutic window between the minimally-effective dose, and maximum tolerable dose in a subject can be determined empirically by a person of skill in the art, with end points being determinable by in vitro and in vivo assays, such as those described herein and/or are acceptable in the pharmaceutical and medical arts for obtaining such information regarding radioprotective agents. Different concentrations of the agents described herein are expected to achieve similar results, with the drug product administered, for example and without limitation, once prior to an expected radiation dose, such as prior to radiation therapy or diagnostic exposure to ionizing radiation, during exposure to radiation, or after exposure in any effective dosage regimen. The compounds can be administered one or more times daily, once every two, three, four, five or more days, weekly, monthly, etc., including increments therebetween. A person of ordinary skill in the pharmaceutical and medical arts will appreciate that it will be a matter of simple design choice and optimization to identify a suitable dosage regimen for prevention, mitigation or treatment of injury due to exposure to radiation.

The compounds described herein also are useful in preventing, mitigating (to make less severe) and/or treating injury caused by radiation exposure. In one embodiment, the radiation is ionizing radiation. Ionizing radiation consists of highly-energetic particles or waves that can detach (ionize) at least one electron from an atom or molecule. Examples of ionizing radiation are energetic beta particles, neutrons, and alpha particles. The ability of light waves (photons) to ionize an atom or molecule varies across the electromagnetic spectrum. X-rays and gamma rays can ionize almost any molecule or atom; far ultraviolet light can ionize many atoms and molecules; near ultraviolet and visible light are ionizing to very few molecules. Microwaves and radio waves typically are considered to be non-ionizing radiation, though damage caused by, e.g., microwaves, may result in the production of free-radicals as part of the injury and/or physiological response to the injury.

### Radiotherapy Protection

### Radiotherapy and Cancer

Radiation therapy works by directing ionizing radiation into the area being treated with the goal of damaging the genetic material of cancerous cells thereby making it impossible for these cells to divide. Accordingly, radiotherapy is an important tool in the fight against cancer and is used in the treatment of as many as 50% of all cancer patients. In fact, more than half a million cancer patients receive radiation therapy each year, either alone or in conjunction with surgery, chemotherapy or other forms of cancer therapy. Other terms for radiotherapy include radiation therapy, x-ray therapy, electron beam therapy, cobalt therapy, or irradiation.

Radiotherapy is especially useful in cases where surgical removal of the cancer is not possible, where surgery might debilitate the patient, or where surgical debulking of the tumor has not absolutely removed all cancerous tissue. Radiotherapy is routinely used following surgery to destroy any cancer cells that were not removed by surgery. Further uses of radiotherapy are prior to surgery where it can “shrink” a previously inoperable tumor down to a manageable size to enable surgical excision.

Radiation therapy can also be used to help relieve symptoms of advanced cancer (such as bleeding or pain), even if a cure is not possible. Over one-third of the practice of radiation therapy is palliative. The typical intent of palliative treatment is to relieve pain quickly and maintain symptom control for the duration of the patient's life. Accordingly, treatment is usually tailored to the patient's clinical condition and overall prognosis. Palliative treatment is often complementary to analgesic drug therapies and may enhance their effectiveness because it can directly target the cause of pain.

Specifically, radiotherapy can be used to treat localized solid tumors, such as cancers of the skin, head and neck, brain, breast, prostate, cervix, and the like. Radiation therapy can also be used to treat cancers of the blood-forming cells and lymphatic system including leukemia and lymphoma respectively, and the like. Mucous membranes or hair in the vicinity of the radiation or in the path of the radiation (e.g., scalp hair in the case of a brain tumor and rectal mucosa in the case of prostate cancer) can be protected using the presently disclosed formulations.

### Radiation Forms and Dosage

External beam radiation therapy commonly uses photons, which are sometimes called “packets of energy,” to treat cancer. It is an object herein to ameliorate the negative effects of all radiotherapy regardless of the form of the photon or particle, including x-rays, gamma rays, UV rays including UV-A, UV-B and UV-C, neutrons, protons, and electrons including beta particles and the like.

X-rays are a very common form of radiation used in radiotherapy. Gamma rays are another form of photons used in radiotherapy. Gamma rays can be produced spontaneously as certain elements (such as radium, uranium, and cobalt 60), which release radiation as they decompose, or decay. Each element decays at a specific rate and can give off energy in the form of gamma rays and other particles. Typically x-rays and gamma rays have the same general effect on cancer cells.

External beam radiation therapy can be delivered by means of a linear accelerator. Typically, linear accelerators use powerful generators to create the high energy rays for external beam radiation therapy. Generally, linear accelerators are capable of producing x-rays at various energies. The linear accelerator can include a special set of lead shutters, called collimators, which focus and direct the rays to the tumor. The linear accelerator can be a large “L-shaped” design which allows it to rotate and deliver radiation from all angles. Multiple angles allow the maximum amount of radiation to be delivered to the tumor while delivering a minimal amount of radiation to the surrounding healthy tissue. The formulations and methods described herein can be used in conjunction with collimators or other devices and methods that limit radiation exposure to normal cells.

Formulations and methods described herein may be capable of ameliorating the effects of most forms of radiotherapy. For example, the compositions and methods can ameliorate the effects of local-field radiation and wide-field radiation. Local field radiation relates to a narrow beam of radiation directed at the specific metastatic site or sites. Customarily, local field radiation has tended to be used for patients with a long life expectancy and fewer metastatic sites. In contrast, wide-field radiation employs a larger field of radiation and is often used to treat patients with a shorter life expectancy and multiple metastatic pain-causing sites.

Radiotherapy dosage is measured by the scientific unit rad (radiation absorbed dose) which is a radiation energy dose equal to an energy of 100 ergs per gram of irradiated material. A patient who receives radiation therapy as a treatment for cancer can receive several thousand rads over a very short period of time (weeks or months). In contrast, a typical scanning x-ray contains far fewer rads. For example, modern mammography systems used to take x-ray images of the breast use approximately 0.1 to 0.2 rad dose per x-ray.

According to traditional radiotherapy, the larger the daily dose of radiation, the lower the total dose that can be administered because of limits to normal tissue tolerance. Proportionately more tumor cells are killed when the daily radiation dose is larger. Typically a balance is obtained between the killing of tumor cells and the adverse radiation effects on normal tissues, which are largely a function of the daily dose. A number of different schedules have been developed that take into account specific tumor characteristics and the tolerance of normal tissues. The literature is divided regarding the optimal radiation schedule to achieve tumor regression and disease palliation of either primary or metastatic sites. Generally, however, radiation treatment is planned in relation to clinical status. Because a main objective herein is to ameliorate the negative effects of radiation therapy, normal tissue can have a higher tolerance to radiation therapy and larger dosages of radiation can be administered safely.

### Side Effects of Radiation

In general, radiation therapy is a local treatment. It typically affects the cells in the treated area. However, as mentioned above, in addition to damaging cancer cells, radiation can also damage normal cells located in the treated area. Normal cells that are located in the treated area can include skin cells, mucous membranes, hair follicles, and the like.

Radiation side effects are typically restricted to the radiation portal and can be classified as either acute, occurring during or immediately after the course of radiation therapy, or late, occurring months to years later. Acute radiation effects are more prominent with radiation schedules that deliver high total doses of radiation with small daily fractions; they generally begin at the end of the second week of therapy. Acute radiation effects, occurring primarily at skin and mucosal surfaces, usually consist of an inflammatory response such as skin erythema or pigmentation, or as mucositis. Late radiation effects may arise without any preceding acute reactions. Fibrosis is the most common type of late radiation injury and can be observed in many types of tissue, including skin.

Other skin conditions caused by radiation therapy include dry and moist desquamation. Dry desquamation, which is characterized by dry and flaky skin and pruritus in the area of irradiation. Moist desquamation, is characterized by sloughing of the epidermis, exposing the moist, raw, dermis layer of the skin.

The rate at which particular hair cells grow is directly proportional to their sensitivity to radiotherapy. Accordingly, the following lists represents particular hair cells' sensitivity to radiotherapy in decreasing order: scalp hair, male beard, eyebrows axilla, pubis, and lastly fine hair. The hair follicle's epithelium is derived from the epidermis and is similarly radiosensitive. As a result, the follicular cells may develop an acute dermatitis, or hyperpigmentation earlier than other cells in the dermis. Hair follicles' sensitivity to radiation can often lead to alopecia in a patient undergoing radiotherapy.

One objective described herein is to ameliorate the negative effects of radiation therapy on normal cells, regardless of whether the effect is acute or late, or whether the effect relates to the patient's skin, mucous membranes, hair follicles, or other treated areas. In one embodiment, the radioprotective formulation described herein is applied topically to the skin at the site of entry of radiation therapy to effect radioprotection of the skin surface.

By the phrase protecting from radiation damage it is implied that relative to damage expected to be incurred to cells, tissue, or organism within a subject or within biological material following exposure to a given amount of radiation (for example ionizing, infra-red or ultra-violet radiation) damage is prevented, minimized or reduced due to effect of the radioprotector compound.

Clinical radiation sources include beam sources (e.g., X-ray, gamma rays, proton beams, etc.) and material sources (e.g., as radium, uranium, cesium 131, cobalt 60, samarium 145, iodine 125 and 127, etc.) that for example may be applied on and/or around a tumor site, or systemically, parenterally, or orally administered.

In certain embodiments methods of cancer radiotherapy or radiosurgery are provided. The methods comprise topically administering to non-tumor skin cells and/or skin tissues in a subject in need of such therapy an amount of a targeted nitroxide agent effective to reduce radiation damage to the non-tumor skin cells and/or skin tissues; and subjecting a tumor or a metastatic cell in the subject to radiation therapy or surgery. The term “cancer radiotherapy” includes radiotherapy involving tumors or lesions, which may be either benign or malignant.

In certain embodiments the biological material (including the human or animal subject) is exposed to the targeted nitroxide agent for a sufficient period of time in advance of anticipated radiation exposure or continuing radiation exposure, such as between about 1 minute and about 3 days, preferably between about 10 minutes and about 6 hours, more preferably between about 20 minutes and about 4 hours and most preferably between about 30 minutes and about 2 hours.

In certain embodiments the formulation disclosed herein is administered preferentially to cells, tissues or organs likely to be exposed to radiation but that are intended to be protected from such radiation exposure. For example, in the case of administration in conjunction with cancer radiotherapy the formulation will preferably be administered preferentially to normal (non-tumor) tissues or cells surrounding a tumor or lesion that are likely to be exposed to radiation in the course of radiotherapy.

In certain embodiments the tumor or neoplasm to be treated is of a cancer selected from the group consisting of lung cancer, colorectal cancer, NSCLC, bronchoalveolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors, and tumor metastasis. In certain embodiments the tumor or tumor metastasis is refractory.

In certain embodiments the radiation is produced by an implanted radiation source and/or by a beam radiation source. In certain embodiments the targeted nitroxide agent is co-administered with an anti-cancer drug. For example, the radioprotective formulations described herein can also be used advantageously in therapy in combination with other medicaments, such as chemotherapeutic agents, for example, radiomimetic agents that are cytotoxic agents that cells, tissues, and/or organs in a manner similar to ionizing radiation. Examples of radiomimetic agents include, but are not limited to bleomycin, doxorubicin, adriamycin, SFU, neocarcinostatin, alkylating agents and other agents that produce DNA adducts.

The formulation may be administered prior to radiation exposure, as well as agents that are effective if administered after irradiation, but before the appearance of symptoms, and agents that are effective if administered after the appearance of symptoms, which may mitigate symptoms or may treat established complications.

### UV Protection

In certain embodiments, the formulations disclosed herein may be used to prevent or treat photo damage, ie the damage induced by UVA/UVB exposure that would be amenable to topical treatment. UVR exerts detrimental effects on human skin by inducing oxidative stress responses via the generation of ROS, which subsequently induce oxidative stress defense responses (reviewed in Photodamage to human skin by suberythemal exposure to solar ultraviolet radiation can be attenuated by sunscreens: a review. Seité S, Fourtanier A, Moyal D, Young A R. Br J. Dermatol. 2010 November; 163(5):903-14. PMID: 20977441). UVA and UVB radiation-induced oxidative stress responses are well known contributors to the pathogenesis of actinic skin damage and photoageing. UVR-induced oxidative stress may be evaluated by quantification of peroxides in proteins or lipids, evaluation of quantity and/or activity of enzymes linked to the skin's antioxidative stress defense response [eg. catalase, GSH, and/or superoxide dismutase (SOD)]. A suberythemal UVA exposure at doses as low as 5-30 J cm is able to induce oxidative stress as measured by hydroperoxides in lipids, or increases of GSH activity.

In certain embodiments, the formulations disclosed herein may be used for prevention and/or therapy of UVA/UVB carcinogenesis that is causative in squamous cell and basal cell carcinoma as well as melanoma. Wavelengths in the UVA (320-400 nm) and UVB (280-320 nm) region of the solar spectrum are absorbed by the skin and produce oxidative stress that contributes to the development of skin cancer (reviewed in Katiyar, S K. Oxidative stress and photocarcinogenesis: Strategies for prevention. Singh, K K., editor. Imperial College Press; London: 2006, p. 933-964. Young A R. Cumulative effects of ultraviolet radiation on the skin: cancer and photoaging. Semin Dermatol 1990; 9:25-31. [PubMed: 2203440], and Punnonen K, Autio P, Kiistala U, Ahotupa M. In-vivo effects of solar-simulated ultraviolet irradiation on antioxidant enzymes and lipid peroxidation in human epidermis. Br J Dermatol 1991; 125:18-20. [PubMed: 1873197]. Acute and/or repeated UVR exposure results in a reduction in the levels of reduced glutathione (GSH), glutathione peroxidase (GPx), and catalase in the exposed skin. Oxidative stress may cause damage at the cellular level, as well as at the molecular level, and this can result in cutaneous inflammation, lipid and protein oxidation, DNA damage, and activation or inactivation of certain enzymes all of which contribute to UVR-induced photodamage of the skin. As used herein, the terms “UV-induced skin damage” or “UV-induced skin disorder” do not refer to acne. These terms are used interchangeably and refer to skin damage resulting from exposure to ultraviolet light in the A (320-400 nm), B (280-320 nm), or C ranges (200-290 nm). Examples of UV-induced skin damage, also referred to herein as “skin lesions”, include wrinkles, hyperpigmentation, dysplasias such as actinic keratosis, and malignant skin tumors such as squamous cell or basal cell carcinoma. The formulations disclosed herein may be used in combination with current sunscreens for example in a block exposure (sunscreens)/prevent damage (antioxidant) synergistic approach.

The formulations disclosed herein may be used in combination with sunscreens for example in a block exposure (sunscreens)/prevent damage (antioxidant) synergistic approach. In certain embodiments, at least one sunscreen agent may be included as an ingredient in the same composition as the targeted nitroxide agent. Illustrative sunscreen agents include, but are not limited to, avobenzone (butyl methoxydibenzoylmethane; commercially available as Parsol 1789 from DSM Nutritional Products, Inc.), oxybenzone (benzophenone-3; commercially available as Neo Heliopan BB from Symrise), and bemotrizinol (bis-ethylhexyloxyphenol methoxyphenyl triazine; commercially available as Tinosorb S from Ciba Corp.), diethylamino hydroxybenzoyl hexyl benzoate (commercially available as Uvinul A Plus from BASF), ethylhexyl triazone (commercially available as Uvinul T150 from BASF), 4-methylbenzylidene camphor (commercially available as Parsol 5000 from DSM Nutritional Products, Inc.), and derivatives and mixtures thereof. Additional non-limiting examples of suitable oil-soluble solid sunscreens are disclosed in The Cosmetic, Toiletry, and Fragrance Association's The International Cosmetic Ingredient Dictionary and Handbook, 10.sup.th Ed., Gottschalck, T. E. and McEwen, Jr., Eds. (2004), p. 2267 and pp. 2292-93. Other sunscreen agents include TiO₂, Fe₂O₃, silica, tin oxide, Cr₂O₃, ZnO, homosalate, octisalate, octocrylene, diethylhexyl 2,6 naphthalate (DEHN), polysilicone-15 (dimethicodiethylbenzal malonate)), bis-disulizole disodium), terephthalylidene dicamphor sulfonic acid, ethylhexyl dimethyl para-amino-benzoic-acid, and ethylhexyl methoxycinnamate.

### EXAMPLES

### Example 1

### Topical JP4-039 Protects Human Skin from Oxidative Skin Damage

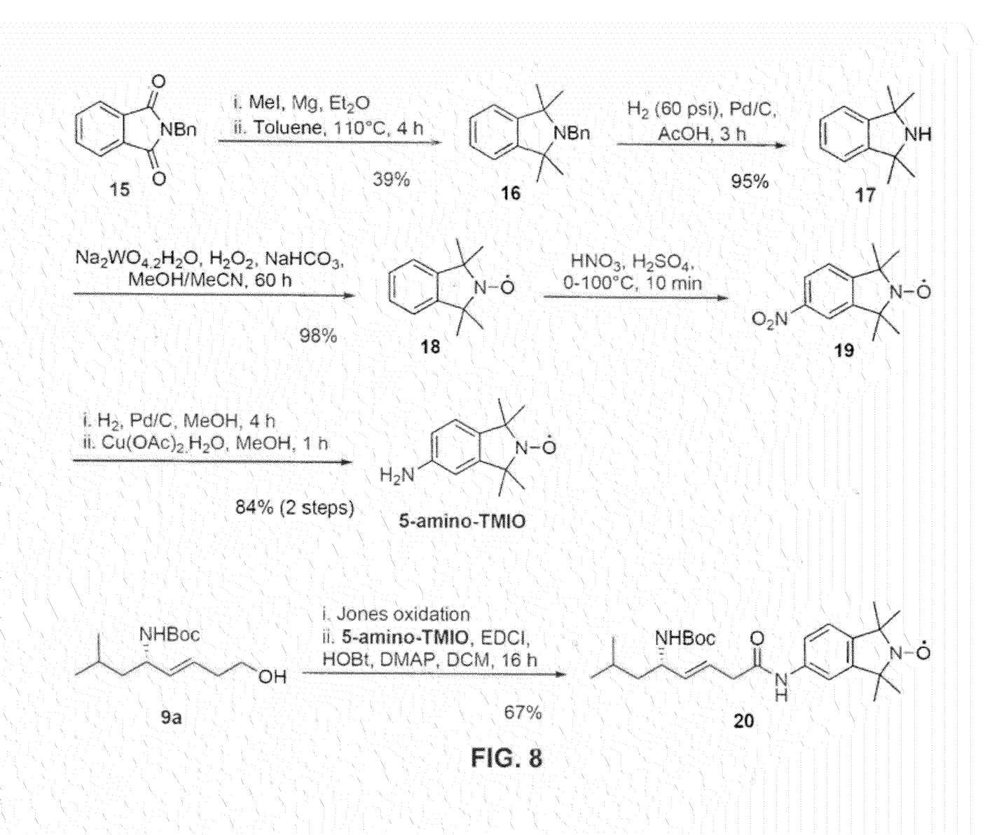

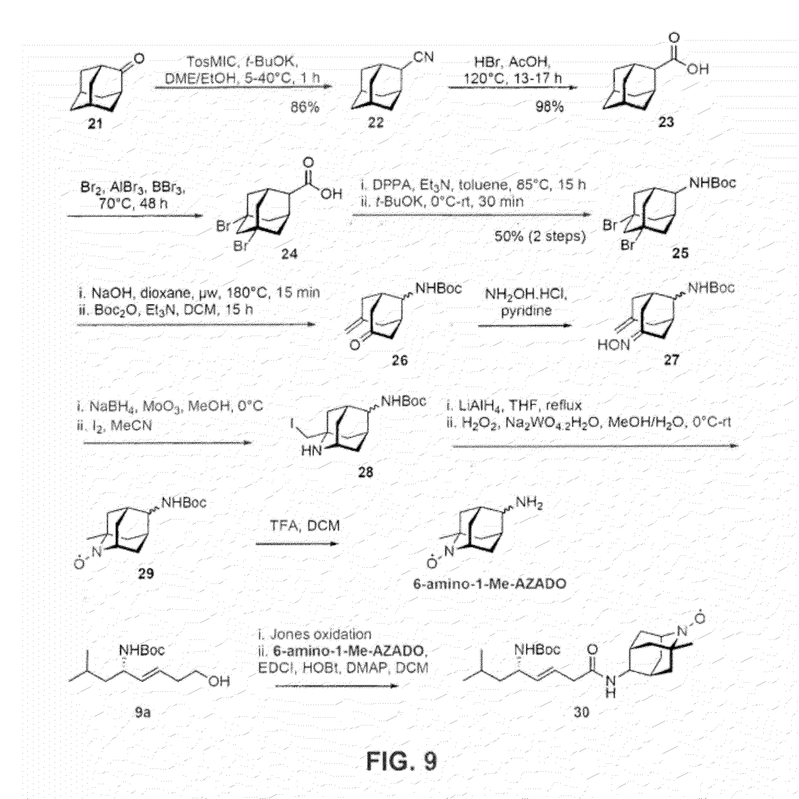

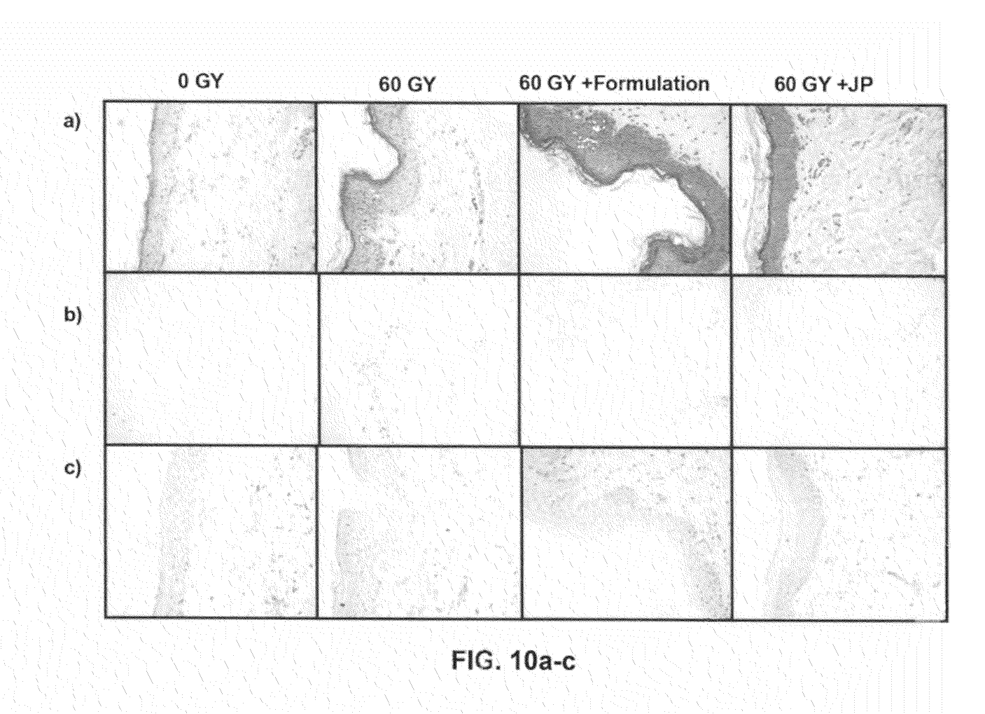

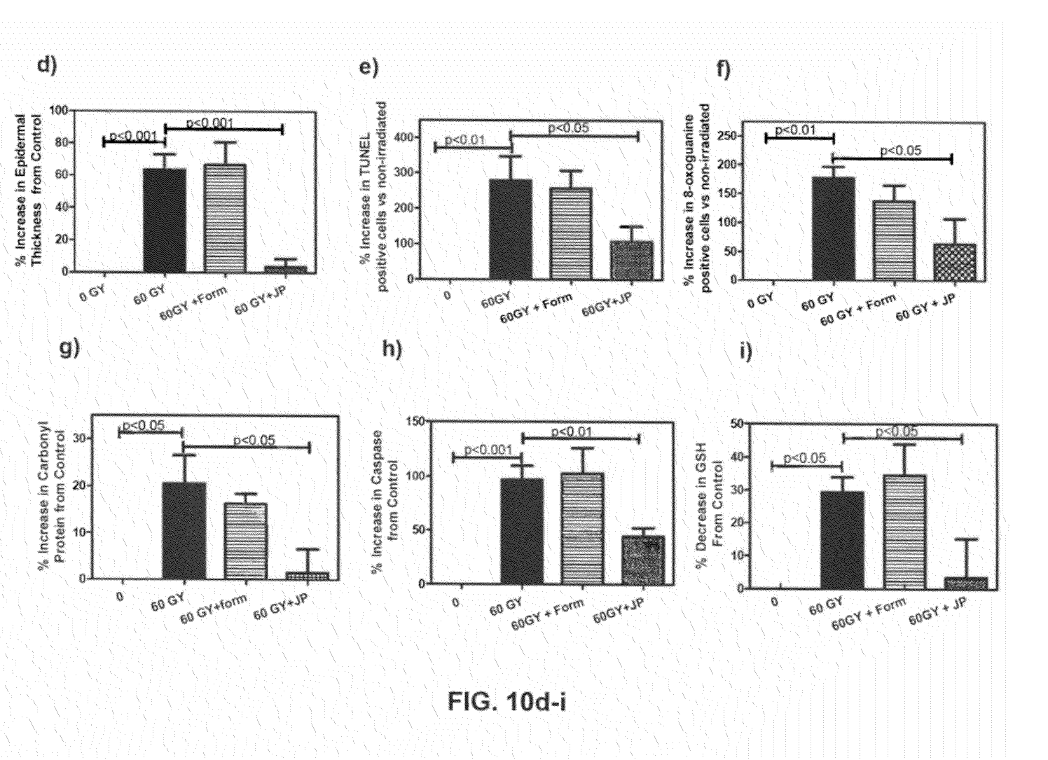

Oxidative skin damage directly correlates with epidermal thickening, an increase in apoptotic (dying) cells (measured by TUNEL and Caspase release) and an increase in 8-oxoguanine positive cells. Further, oxidative damage is a result of free radical formation that correlates with increases in carbonylated proteins and reduced levels of GSH. Using all of these histopatholgic and enzymatic measures of oxidative damage and free radical formation related to cell death, these results demonstrate that topically applied JP4-039 protects human skin from oxidative damage that has been correlated with a wide variety of human skin pathologies including oncogenesis and skin aging. Living human skin samples were divided and groups of skin samples were treated with either 60GY iradiation (60GY), 60GY followed by topical JP4-039 (60GY+JP4-039), 60GY followed by formulation without JP4-039 (60GY+Formulation), or were cultured without irradiation (0 GY CONTROL). Photographs in FIG. 10 a) are representative H&E sections (400×). FIG. 10 b) shows representative TUNEL stained sections with blue TUNEL positive cells and red negative cells (400×). In FIG. 10 c) sections were stained with antibody against 8-oxoguanine followed by DAB and counterstained with hemotoxalin. Positive cells have brown nuclei (400×). Epidermal thickness FIG. 10 (d) was determined by photographing 5 consecutive H&E sections (400×) and measuring the length of the epidermis at 5 sites within each photograph. The percentage of TUNEL positive cells FIG. 10 (e) and 8 oxoguanine positive cells FIG. 10 (f) was determined by counting all epidermal cells (positive and negative) in five consecutive 400× sections beginning with the site of maximal apoptosis. Carbonyl Protein FIG. 10 (g), Caspase 3/8 FIG. 10 (h) and GSH FIG. 10 (i) levels were determined in skin homogenates and were normalized by mg protein. Carbonyl protein samples were performed in triplicate from pooled homogenates. All graphical data were converted to % increase from control and are presented as mean±sem. Statistical significance was determined by ANOVA followed by a Bonferroni post test with significance set at p<0.05.

### Example 2

### JP04-39 is a potent topical antioxidant that can mitigate skin damage from ionizing radiation

Mouse flank was shaved and depilated 24 hrs. prior to irradiation with 35 GY using a 6 MeV electron beam.

Clinical Effect—21 Day Experiments:

JP4-039 was topically applied in a liposome formulation (F14-JP4-039 Emulsion: JP4-039 (2 mg/ml) was emulsified in sesame seed oil (100 mg/ml), and soy phosphatidyl choline (50 mg/ml) by sonication) 30 minutes after irradiation and daily for the next 3 days. Mice were photographed and skin damage scored by clinical criteria. Leg contracture was measured by comparing maximal extension between control and irradiated legs. Histological characterization of cellular infiltrates and collagen levels was performed.

Medical Effect—4 Hour Studies:

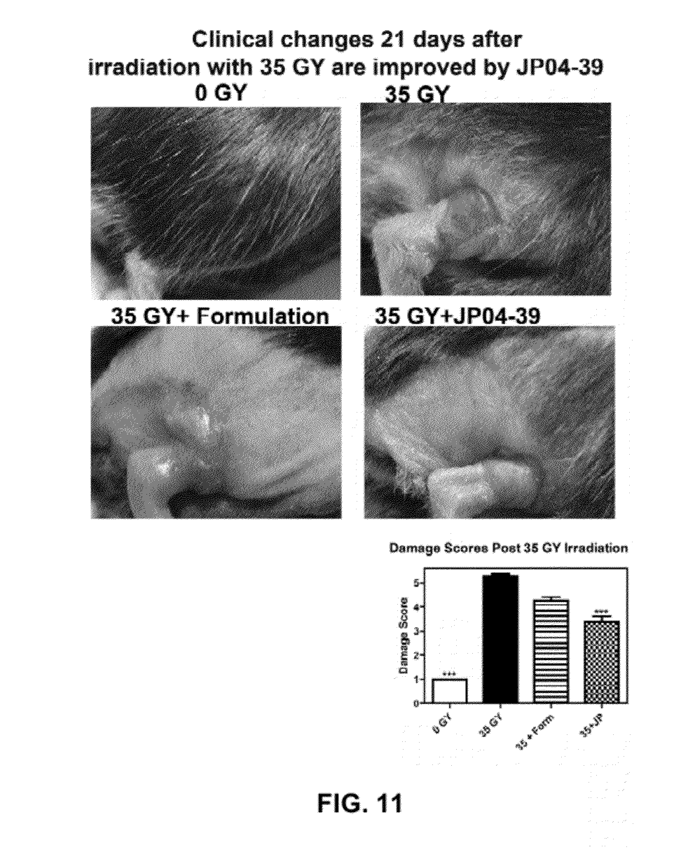

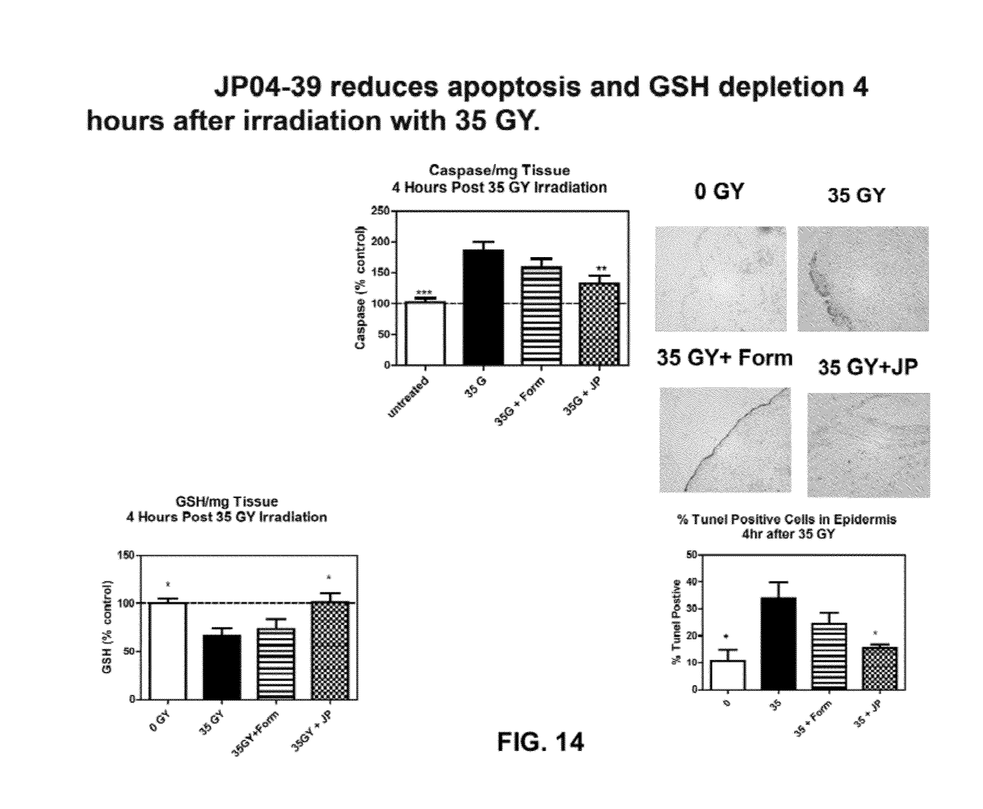

JP4-039 was topically applied in a the above-described liposome formulation 30 minutes after irradiation. Skin samples taken at 4 hours. Analyzed for apoptosis via caspase 3 and TUNEL. Antioxidant functions evaluated by changes in GSH levels. All quantitative data presented as means±sem and significance determined by ANOVA followed by a Bonferoni post test set at p<0.05. The results are shown in FIGS. 11-14.

Taken together, these studies revealed that topically applied JP04-39 mitigates clinically evident acute skin damage, and damage mitigation correlates with reductions in cellular infiltrates. Topically-applied JP04-39 also demonstrated potent antioxidant function and prevented radiation-induced apoptosis in the skin. These results suggest that topically applied JP04-39 is a potent antioxidant that can prevent/reverse acute irradiation damage.

Materials/Methods: The right legs of C57BL/6NHsd mice were shaved and treated with Nair to remove the fur. Twenty four hours later the right legs were irradiated to doses of 30 or 35 Gy using 6 MeV electron beam, bolused to bring the surface dose to 100%. At 30 minutes after irradiation, 24, 48 and 72 hours, F14 emulsion only or F14-JP4-039 (100 μg in 50 μl) was applied to the skin. Photos were taken for visual evidence of skin damage and leg contracture was measured as a functional evaluation of tissue damage. Histological characterization of skin thickening, cellular infiltrates and collagen evaluation was also performed. To evaluate the mechanism of radiation damage mitigation, skin samples were assayed at 4 hours after irradiation for markers of apoptosis and oxidative stress.
Results: Skin damage was scored on day 21 after irradiation. Damage after 35 Gy was reduced significantly after treatment with F14-JP4-039 (3.4±0.2) compared to irradiation controls and irradiated mice treated with F14 only (5.3±0.1 and 4.3±0.2, p<0.01). Reduced leg contracture caused by radiation was reversed in irradiated legs treated with F14-JP4-039, with the difference between irradiated and control leg at 5.3±0.8 cm compared to irradiation only or irradiated F14 treated leg at 9.2±1.0 or 10.4±0.5 cm, respectively, p<0.01. These results were consistent with increases in collagen levels observed with Mason's Trichrome staining. The F14-JP4-039 treated skin showed less inflammation as evidenced by reduced cellular infiltrate observed with H&E staining (32.3±2.3) compared to control irradiated skin (45.0±2.6 p<0.01) and F14 treated irradiated skin (50.4±2.8, p<0.01). There was also a significantly decreased apoptosis 4 hours after irradiation in skin treated with F14-JP4-039 (15.5±4.2% of cells) compared to the control and F14 treated mice (35.0±5.8 or 24.2±4.2%, respectively, p<0.05).
Conclusions: Topically applied JP4-039 mitigates irradiation induced skin damage, and mitigation correlates with reductions in cellular infiltrates and fibrotic contractures. Topically applied JP4-039 is a potent mitigator of acute irradiation skin damage.
Mouse Model: The rear leg of female C57BL/6NHsd mouse was shaved and treated with Nair to remove the fur. Twenty-four hours later the mice were anesthesized and the rear leg was irradiated to either 30 or 35 Gy using a 6 MeV electron beam with a bolus placed on the skin to bring the maximum dose to the skin. Thirty minutes, 24 hr, 48 hr and 72 hr later the skin was treated with F14 emulsion only or F14 emulsion containing JP4-039 by applying 50 μl of the emulsion to the skin and spreading it over the exposed skin. This will deliver a dose of 200 μg of JP4-039 to the skin. The mice were sacrificed 4 hr or 21 days after irradiation.
Assessment of Irradiation Damage: Skin damage was measured using visual evidence seen from photographs of the skin and measuring leg contracture. The skin on the right leg was then removed and cut into three sections. One was frozen on dry ice, one section fixed in formalin and one section frozen in OCT (Optimum Cutting Temperature). Histological sections were characterized by skin thickening, cellular infiltrates and collagen evaluation. The mechanism of mitigation of irradiated skin damage was performed by analysis of apoptosis and oxidative stress.
Application of a F14 emulsion containing the mitochondrial targeted nitroxide JP4-039 at 30 min after irradiation resulted in a significant decrease in irradiation damage as determined by decreased visual scoring of the irradiation damage, decreased thickening of the skin, decreased number of cellular inflitrates, decreased differential leg extension and decreased caspase 3 activity and apoptosis.

### Example 3

### Topical and Transdermal Absorption of the GS-Nitroxides XJB-5-125 and JP4-039

Initially we sought to characterize the absorption/penetration of a topically applied representative GS-Nitroxide XJB-5-125 in mouse skin. The physical properties of a chemical are critical to its ability to penetrate into and through the skin. The two most important factors are the log octanol/water (Ko/w) partition coefficient and the molecular weight. For XJB-5-125, the log Ko/w=4.5 and molecular weight is 956. The lipophilicity “rule” is based on the need for a compound to partition out of the lipophilic stratum corneum and into the more hydrophilic epidermis and dermis (54).

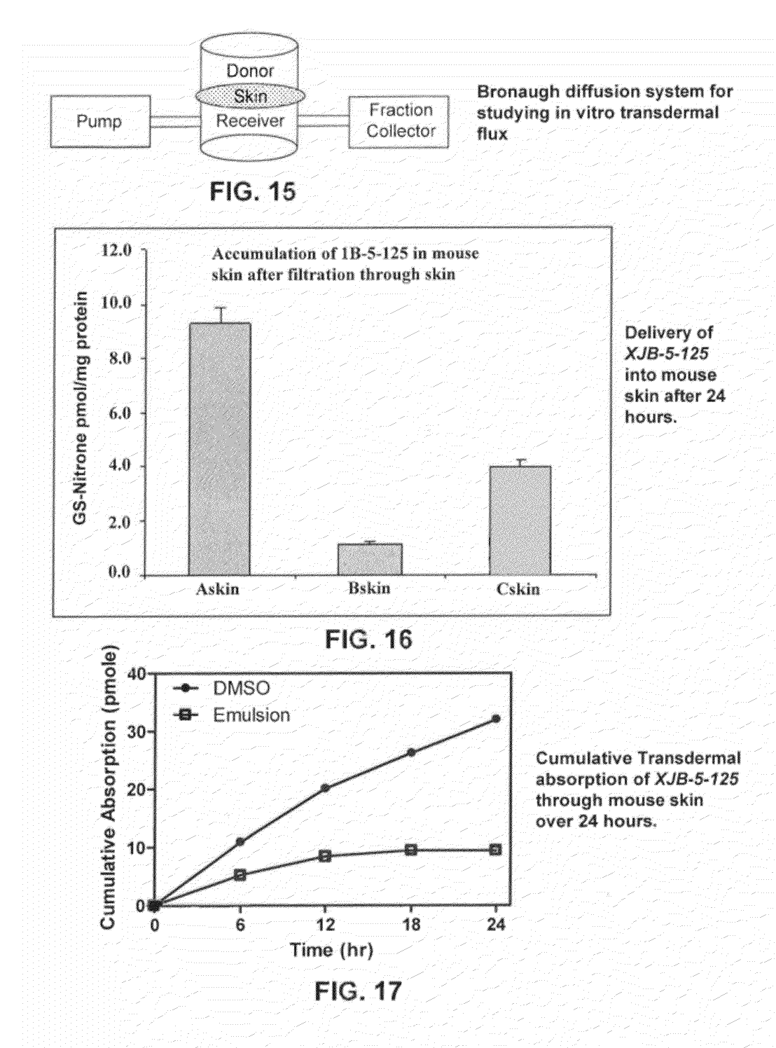

To evaluate XJB-5-125 penetration in mouse skin, C57/BL6 mice were shaved using animal clippers (#40 blade), followed by a brief treatment with Nair (depilatory) to remove remaining hair. The skin was washed immediately after hair removal to prevent further irritation, and then was allowed to recover for 24 hours prior to study. This reduces interference by hair and allows time for small abrasions to heal prior to dermal penetration studies. A small piece of skin (2 cm²) was placed in a Bronaugh style flow-through diffusion cell system (PermeGear, Riegelsville Pa.) (FIG. 15). It was then sandwiched between two pieces of the inert polymer Kel-F and clamped shut to prevent leakage. The epidermal side faces upward and is exposed to the donor solution (test solution), and the dermal side is in contact with the receptor fluid. The exposed surface area is 0.79 cm² (circular chamber with 1 cm diameter). The skin forms a water-tight seal in the flow through chamber so the receiving fluid (PBS+25% ethanol) on the dermal side will contain the XJB-5-125 only if it has penetrated through the skin. The receiver chamber was perfused with this buffer that then passes to a fraction collector via Teflon tubing. The PBS+25% ethanol was used because it is an effective sink for hydrophobic compounds and produces better in vitro/in vivo correlations than other receiver solutions. The skin was maintained at 32 C by placing the chamber in a metal block heated via a recirculating water bath. The skin was equilibrated for 60 minutes prior to introduction of the test compound. Seventy five μL of XJB-5-125 was placed on the skin and was allowed to remain for the course of the experiment. The efflux was collected for 24 hours.

Upon completion of the study the skin was removed from the diffusion chamber. The stratum corneum, which will contain the majority of the topically applied compound, but is not relevant from a therapeutic standpoint, was removed by sequential tape-stripping (15 times) using Bookman Tape (3M, Minneapolis, Minn.). The remaining skin (viable epidermis and dermis) and transdermal effluent were assayed for XJB-5-125 via ESR.

Mouse skin was homogenized in 400 μl 50 mM PBS pH 7.4. EPR measurements were performed in gas-permeable Teflon tubing (0.8 mm internal diameter, 0.013 mm thickness) obtained from Alpha Wire Corp. (Elizabeth, N.J., USA) on a JEOL JES-RE I X spectrometer at 25° C. The Teflon tube (approximately 8 cm in length) was filled with 70 μl of sample containing 28.5% of acetonitrile and 2 mM K3Fe(CN)₆, folded in half, and placed into an open EPR quartz tube (inner diameter of 3.0 mm). EPR spectra were recorded at 334.7 mT, center field; 20 mW, power; 0.079 mT, field modulation; 5 mT, sweep width; 400 and 4000, receiver gain; 0.1 s, time constant. Spectra were collected using EPRware software (Scientific Software Services, Bloomington, Ill., USA).

This preliminary experiment demonstrates that XJB-5-125 can sufficiently penetrate intact skin. We determined both the level of XJB-5-125 present in the viable skin and the total transdermal absorption of drug after 24 hours (FIGS. 16,17).

Donor A=1 mM XJB-5-125 in DMSO,

Donor B=1 mM XJB-5-125 in 95% Propylene Glycol+5% Linoleic Acid, and

Donor C=1 mM XJB-5-125 in 50% ETOH+40% H2O+5% Propylene Glycol+5% Brij30.

A total of 75 nmole was placed on top of each section of skin to begin these experiments (75 μl of 100 mM). The delivery of XJB-5-125 into the skin resulted in 0.46% remaining within the skin after 24 hours. The higher delivery rate observed is within the range of other topical products.

Given the observation that XJB-5-125 is active in cells in the concentration range from 2.5-20 μM and assuming a tissue density of 1 g/cm³, an order of magnitude analysis based on these data demonstrates topical delivery of XJB-5-125 as a skin therapeutic agent. Additionally, the fact that the total skin absorption is generally regarded as linearly related to the donor concentration implies that topical delivery will be greatly enhanced by increasing the donor concentration.

We analyzed delivery of JP4-039 with similar results. Further, we extended these results by analyzing topical delivery of JP4-039 in vivo using a novel lipid-based formulation for delivery. For these studies, 2 mg/ml JP4-039 was formulated in saline with a mixture of surfactants with egg PC by sonication to a liposome size of 200 nm. Harvested, tape-stripped (to remove superficial drug) skin from topically treated mice was processed as described above to determine the amount of JP4-039 retained in the skin. We found cutaneous retention to be in the range of 11.7+/−3.1 pmole/mg, a level compatible with therapeutic efficacy. These studies demonstrate that these agents can be delivered topically to obtain biologically relevant levels in the skin.

### Example 4

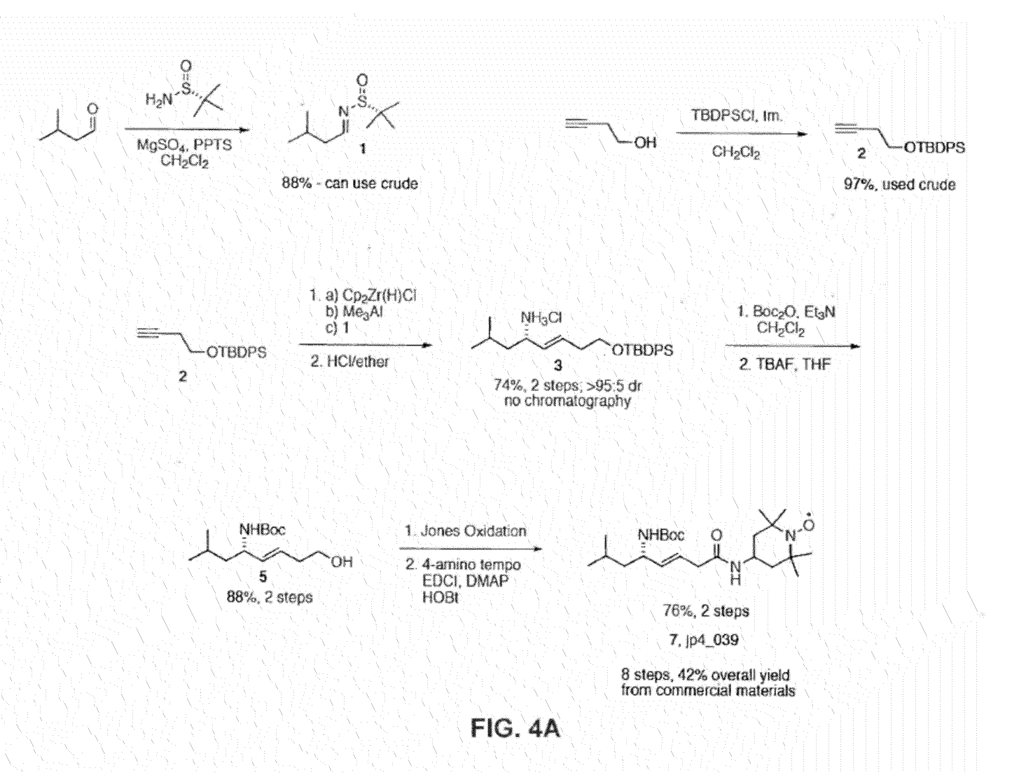

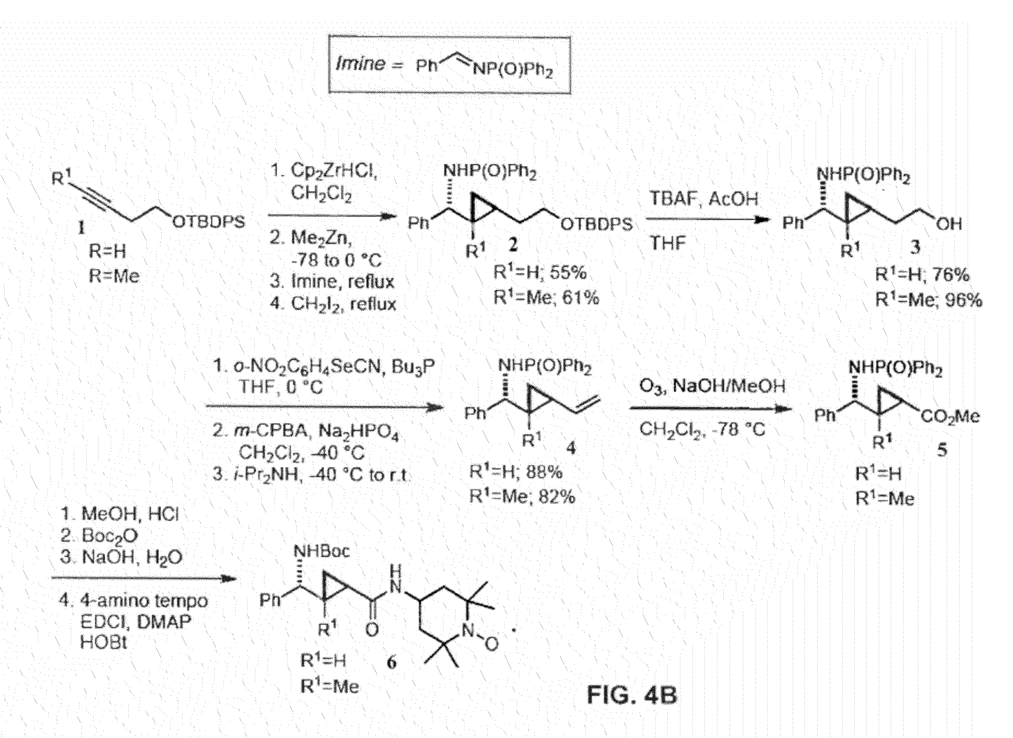

### Synthesis of JP4-039 (see FIG. 4)

Synthesis of JP4-039 was accomplished according to the following.

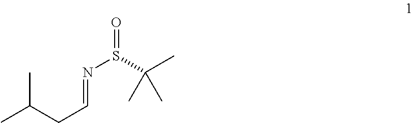

(R,E)-2-Methyl-N-(3-methylbutylidene)propane-2-sulfinamide (1) (Staas, D. D.; Savage, K. L.; Homnick, C. F.; Tsou, N.; Ball, R. G. J. Org. Chem., 2002, 67, 8276)—To a solution of isovaleraldehyde (3-Methylbutyraldehyde, 5.41 mL, 48.5 mmol) in CH₂Cl₂ (250 mL) was added (R)-2-methylpropane-2-sulfinamide (5.00 g, 40.4 mmol), MgSO₄ (5.0 eq, 24.3 g, 202 mmol) and PPTS (10 mol %, 1.05 g, 4.04 mmol) and the resulting suspension was stirred at RT (room temperature, approximately 25° C.) for 24 h. The reaction was filtered through a pad of Celite® and the crude residue was purified by chromatography on SiO₂ (3:7, EtOAc:hexanes) to yield 6.75 g (88%) as a colorless oil. ¹H NMR δ 8.07 (t, 1H, J=5.2 Hz), 2.47-2.38 (m, 2H), 2.18-1.90 (m, 1H), 1.21 (s, 9H), 1.00 (d, 6H, J=6.7 Hz). As an alternative, filtration through a pad of SiO₂ provides crude imine that functions equally well in subsequent reactions.

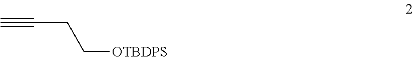

(But-3-ynyloxy)(tert-butyl)diphenylsilane (2) (Nicolaou, K. C.; Lizos, D. E.; Kim, D. W.; Schlawe, D.; deNoronha, R. G.; Longbottom, D. A.; Rodriquez, M.; Bucci, M.; Cirino, G. J. Am. Chem. Soc. 2006, 128, 4460)—To a solution of 3-butyn-1-ol (5.00 g, 71.3 mmol) in CH₂Cl₂ (400 mL) was added imidazole (5.40 g, 78.5 mmol) and TBDPSCl ((tert-butyl)diphenylsilane chloride)(22.0 g, 78.5 mmol) and the reaction was stirred at RT for 22 h. The reaction was filtered through a pad a SiO₂, the SiO₂ washed with CH₂Cl₂ and the colorless solution concentrated to yield 21.4 g (97%) of crude alkyne that was carried on without further purification.

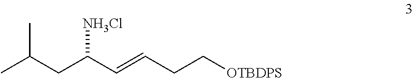

(S,E)-8-(tert-Butyldiphenylsilyloxy)-2-methyloct-5-en-4-amine hydrochloride (3)—To a solution of (2) (15.9 g, 51.5 mmol) in CH₂Cl₂ (300 mL) was added zirconocene hydrochloride (15.1 g, 58.4 mmol) in 3 portions and the resulting suspension was stirred at RT for 10 min. The resulting yellow solution was cooled to 0° C. and Me₃Al (2.0 M in hexanes, 27.5 mL, 54.9 mmol) was added and stirred for 5 minutes followed by addition of a solution of imine (1) (6.50 g, 34.3 mmol) in CH₂Cl₂ (50 mL) and the orange solution was stirred for an additional 4 h while allowed to warm to rt. The reaction was quenched with MeOH, diluted with H₂O and CH₂Cl₂ and HCl (1 M) was added to break up the emulsion (prolonged stirring with Rochelle's salt can also be utilized). The organic layer was separated and the aqueous layer was washed with CH₂Cl₂ (2×). The organic layers were combined, washed with brine, dried (MgSO4), filtered though a pad of Celite® and concentrated. Since the crude oil was contaminated with metal salts, the oil was dissolved in Et₂O (diethyl ether, Et=ethyl), allowed to sit for 2 h, and then filtered though a pad of Celite® and concentrated. Analysis of the crude residue by 1H NMR showed only 1 diastereomer (>95:5 dr).

To the crude residue in Et₂O (800 mL) was added HCl (4.0 M in dioxane, 17.2 mL, 68.7 mmol) and the reaction was stirred for 30 minutes, during which time a white precipitate formed. The precipitate was filtered, washed with dry Et₂O, and dried to afford 11.0 g (74% over 2 steps) of (3) as a colorless solid: [α]_{D}−2.9 (c 1.0, CH2Cl2); ¹H NMR δ 8.42 (bs, 3H), 7.70-7.55 (m, 4H), 7.48-7.30 (m, 6H), 5.90 (dt, 1H, J=14.9, 7.5 Hz), 5.52 (dd, 1H, J=15.4, 8.4 Hz), 3.69 (appt, 3H, J=6.5 Hz), 2.45-2.20 (m, 2H), 1.80-1.50 (m, 3H), 1.03 (s, 9H), 0.95-0.84 (m, 6H); ¹³C NMR δ 135.5, 134.5, 133.7, 129.5, 127.6, 127.3, 63.0, 52.9, 42.1, 35.6, 26.7, 24.4, 22.9, 21.5, 19.1; EIMS m/z 395 ([M-HCl]⁺, 40), 338 (86), 198 (100); HRMS (EI) m/z calcd for C₂₅H₃₇NOSi (M-HCl) 395.2644. found 395.2640.

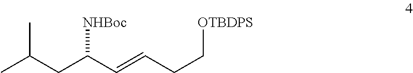

(S,E)-tert-Butyl 8-(tert-butyldiphenylsilyloxy)-2-methyloct-5-en-4-ylcarbamate (4)—To a solution of (3) (10.5 g, 24.3 mmol) in CH₂Cl₂ (400 mL) was added Et₃N (triethylamine) (3.0 eq, 10.3 mL, 72.9 mmol) and Boc₂O (1.05 eq, 5.74 g, 25.5 mmol) and the resulting suspension was stirred at RT for 14 h. The reaction was quenched with sat. aq. NH₄Cl, the organic layers separated, dried (MgSO₄), filtered and concentrated. The crude residue was carried onto the next step without further purification.

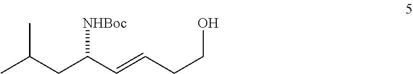

(S,E)-tert-Butyl 8-hydroxy-2-methyloct-5-en-4-ylcarbamate (5)—To a solution of crude (4) (12.0 g, 24.3 mmol) in THF (200 mL) at 0° C. was added TBAF (1.0 M in THF, 1.25 eq, 30.4 mL, 30.4 mmol) and the reaction was warmed to RT and stirred for 2 h. The reaction was quenched with sat. aq. NH₄Cl, organic layer washed with brine, dried (MgSO₄), filtered and concentrated. The crude residue was purified by chromatography on SiO₂ (3:7, EtOAc:hexanes) to yield 5.51 g (88%, 2 steps) as a colorless oil. [α]_{D}−12.7 (c 1.0, CH₂Cl₂); ¹H NMR δ 8.08 (t, 1 H, J=4.1 Hz), 7.40-7.15 (m, 20H), 4.79-4.41 (m, 8H), 4.05-3.95 (m, 3H), 3.89-3.79 (m, 1H), 3.79-3.70 (m, 2H), 3.70-3.61 (m, 1H), 2.70-2.52 (m, 1H), 2.50-2.36 (m, 1H), 2.04-1.80 (m, 2 H); ¹³C NMR δ 168.7, 138.1, 138.0, 137.9, 137.8, 128.0, 128.0, 127.9, 127.6, 127.5, 127.4, 127.2, 127.2, 127.1, 76.2, 73.8, 72.8, 72.8, 72.6, 72.1, 69.9, 67.1, 56.0, 32.1, 22.9, 21.9; EIMS m/z 257 ([M]⁺, 10), 227 (55), 171 (65); HRMS (EI) m/z calcd for C₁₄H₂₇NO₃ 257.1991. found 257.1994.

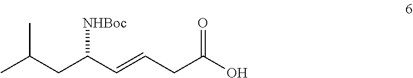

(S,E)-5-(tert-Butoxycarbonylamino)-7-methyloct-3-enoic acid (6)—To a solution of (5) (1.00 g, 3.89 mmol) in acetone (40 mL) at 0° C. was added a freshly prepared solution of Jones Reagent (2.5 M, 3.89 mL, 9.71 mmol) and the reaction was stirred at 0° C. for 1 h. The dark solution was extracted with Et₂O (3×50 mL), the organic layers washed with water (2×75 mL), brine (1×50 mL), dried (Na₂SO₄), filtered and concentrated to yield 990 mg (94% crude) of acid (6) as a yellow oil that was used without further purification.

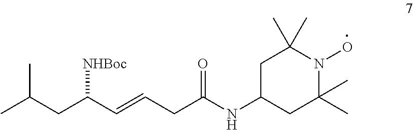

(S,E)-5-(tert-Butoxycarbonylamino)-7-methyloct-3-enoic acid-TEMPO (7)—To a solution of (6) (678 mg, 2.50 mmol, crude) in CH₂Cl₂ (35 mL) at 0° C. was added 4-amino tempo (1.5 eq, 662 mg, 3.75 mmol), EDCI (1.2 eq, 575 mg, 3.00 mmol), DMAP (1.1 eq, 339 mg, 2.75 mmol) and HOBt-hydrate (1.1 eq, 377 mg, 2.75 mmol) and the resulting orange solution was stirred at RT for 14 h. The reaction was diluted with CH₂Cl₂, washed with sat. aq. NH₄Cl and the organic layer dried (Na₂SO₄), filtered and concentrated. The crude residue was purified by chromatography on SiO₂ (1:1 to 2:1, EtOAc/hexanes) to yield 857 mg (76%, 2 steps) as a peach colored solid. Compound purity determined by LCMS and ¹H NMR.

The compounds shown as Formula 4, above can be synthesized as shown in FIG. 4. Briefly, synthesis was accomplished as follows: To a solution of compound (I) in CH₂Cl₂ was added zirconocene hydrochloride, followed by addition of Me₂Zn, then a solution of N-diphenylphosphoryl-1-phenylmethanimine (Imine). The reaction mixture was refluxed, filtered, washed, and dried to afford (2). Cleavage of the TBDPS protecting group was achieved by treating (2) with TBAF, which resulted in the formation of (3). The terminal alcohol (3) was dehydrated to alkene (4), which was further treated by ozonolysis to afford ester (5). Protocols similar to that given for the synthesis of JP4-039, above, were used to acylate the amino group with the Boc protecting group and to react the terminal carboxylic acid with 4-amino-TEMPO to afford (6).

### Example 5

### Topically Applied Targeted Nitroxides Mitigate Oxidative Stress Induced Skin Damage—Barrier Function, Fibrosis, Thickening

Clinically relevant disruption of skin barrier function results in increased water loss from the skin surface. This can be measured non-invasively by the Transepidermal Water Loss (TEWL) assay. Increasing TEWL values directly correlate with damage to the skin's barrier function.

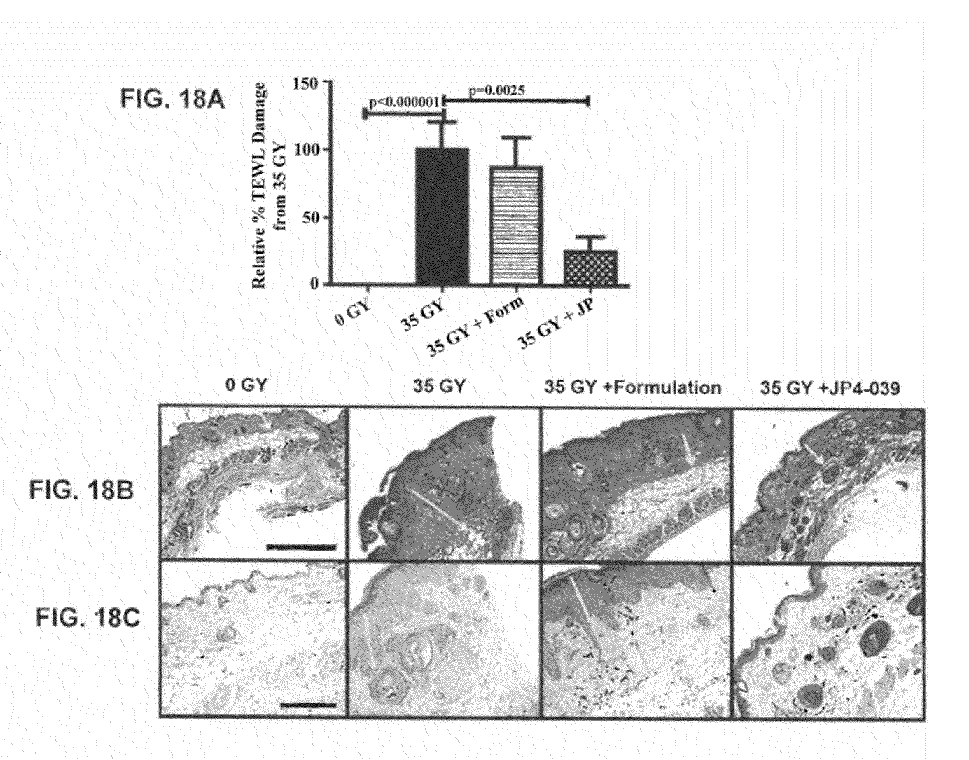

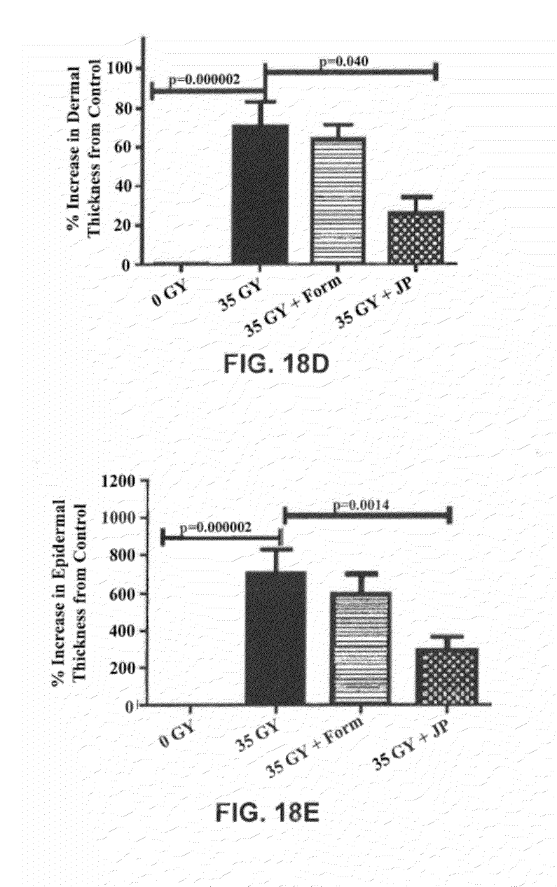

Groups of mice were irradiated with 35 GY to induce oxidative stress and then treated with JP4-039 or formulation alone 0.5, 24 and 48 hours after exposure. Skin was evaluated 21 days after initial treatment. Damage to skin barrier function was determined by TEWL. Results are reported as % recovery from 35GY damage defined as (((radiated average−test sample value+0GY average)/(treated average)*100) (FIG. 18a).

Our results demonstrate that irradiation with 35 GY increased TEWL levels compared to 0 GY exposure (FIG. 18). Importantly, treatment with topical JP4-039 significantly reversed (p=0.011) the radiation induced TEWL increases reflecting prevention/mitigation of radiation induced skin barrier damage.

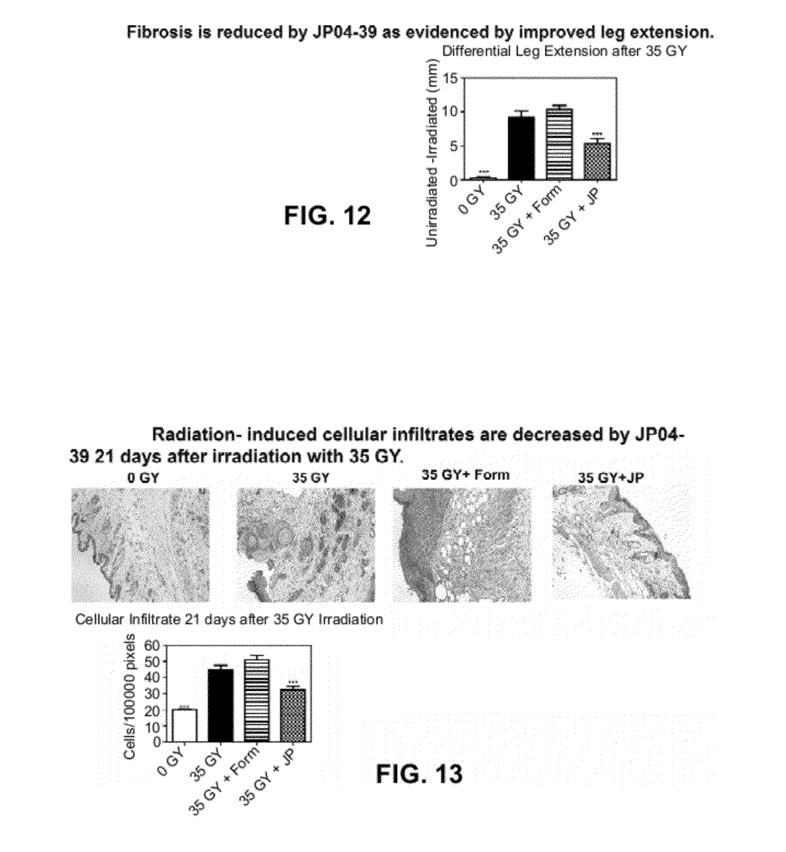

Data presented in FIG. 12 demonstrates that topical JP4-039 prevents/mitigates radiation induced clinical fibrosis as measured by leg extension. Oxidative stress induced skin fibrosis is associated with increases in skin collagen levels and dermal and epidermal thickening. To determine the capacity of JP4-039 to prevent/mitigate these effects, we examined histological sections of treated and untreated skin using Masson's Trichrome stain to identify collagen.

Fibrosis was evaluated histologically by evaluating the extent of collagen infiltration in the skin sections in (FIG. 18b) (100×). Dermal thickness is indicated by the arrows and collagen fibers were identified by Masson's Trichrome staining (blue). Representative H&E stained sections are provided in (FIG. 18c) with arrows showing epidermal thickness (200×). Dermal (FIG. 18d 100×) and epidermal (FIG. 18e) thickness (200×) was quantified at 5 sites within 4 representative histologic images for each mouse using NIH Image J Software. Statistical significance was determined by ANOVA followed by a Bonferroni post test set at p<0.05.

Our results indicate that topically applied JP4-039 prevents/mitigates fibrosis associated changes including epidermal and dermal thickening, and increased collagen deposition (FIG. 18).

### Example 6

### Topically Applied Targeted Nitroxides Mitigate Oxidative Stress Induced Skin Damage Even when Applied 24 Hours after Exposure to Ionizing Radiation

To extend our results further, we performed experiments to determine if topically targeted nitroxides could mitigate oxidative stress induced skin damage even when applied long after the stress event. To accomplish this, we irradiated animals as described previously, but withheld application of JP4-039 until 24 h after the stress event.

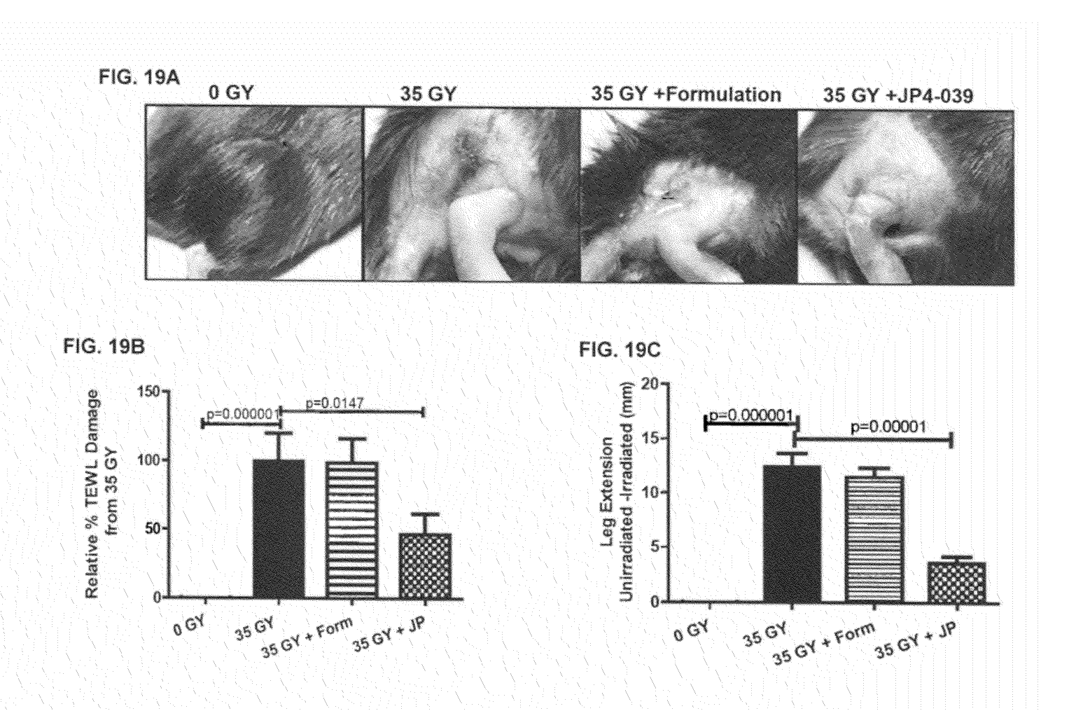

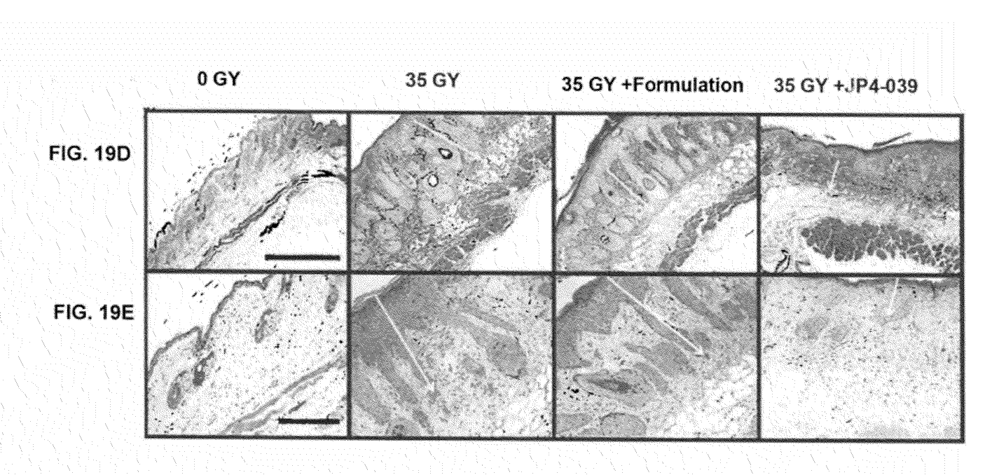

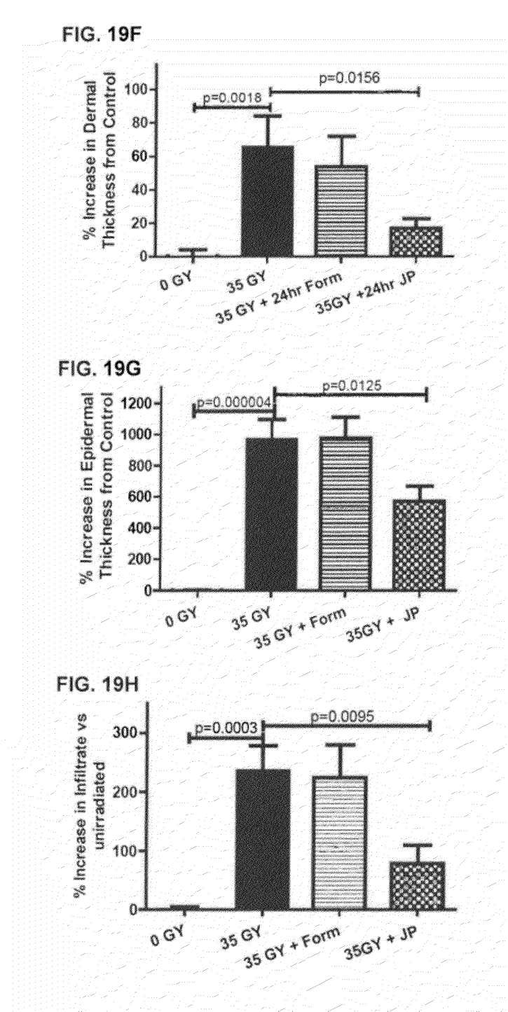

C57/BL6 mice were irradiated with 35 GY and treated with JP4-039 or formulation alone starting 24, 48 and 72 hours after exposure. Skin was evaluated 21 days after initial treatment. Representative clinical photographs at the 21 day time point are shown for each treatment condition (FIG. 19a). Damage to skin barrier function was determined by TEWL. Results are reported as % recovery from 35GY damage defined as (((radiated average—test sample value+0GY average)/(treated average)*100) (FIG. 19b). As a clinical measure of fibrosis, leg extension was determined and is reported as the difference between treated and untreated legs (FIG. 19c). Fibrosis was confirmed histologically by evaluating the extent of collagen infiltration in the skin sections (FIG. 19d) (100×). Dermal thickness is indicated by the arrows and collagen fibers were identified by Masson's Trichrome staining (blue). Representative H&E sections are provided in (FIG. 19e) with arrows showing epidermal thickness (200×). Dermal (FIG. 19f) (100×) and epidermal (FIG. 19g) thickness (200×) were quantified at 5 sites within 4 (dermal) or 5 (epidermal) representative histological images for each condition using NIH Image J software. The results are reported as % increase from control using the formula (((treated/untreated average)−1)*100). Cellular infiltrates (FIG. 19h) were determined by drawing a 100,000 pixel box in the dermis (NIH Image J software) and counting cellular infiltrate in each box. All data are presented as mean±sem (n=5-20). Statistical significance was determined by ANOVA followed by a Bonferroni post test set at p<0.05.

The results of these studies demonstrate that the application of topical targeted nitroxides well after a stress event mitigates skin damage, and this mitigating effect is comparable to that observed in the setting of early application (FIG. 19).

### Example 7

### Synthesis of Additional Targeted Nitroxide Agent

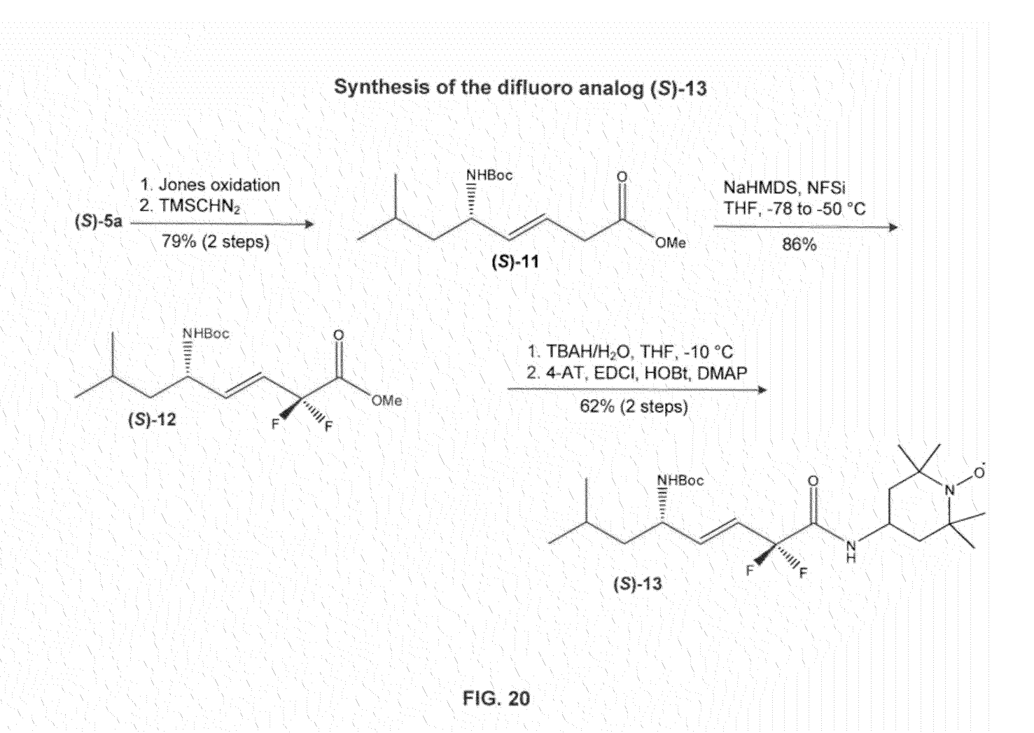

A difluorinated analog of JP4-039 was envisioned to enhance the bioavailability of the agent as shown in the synthesis scheme of FIG. 20. The methyl ester (5)-11, prepared from alcohol (S)-5a, was treated with 3 equiv. of the fluorinating agent N-fluoro-N-(phenylsulfonyl)benzenesulfonamide (accufluor, NFSi) and 2.3 equiv. of NaHMDS in THF at −78° C. to afford the desired difluoroester (S)-42 in 86% yield (Scheme 6). Saponification with tetra-butylammonium hydroxide (TBAH) and condensation with 4-AT provided the difluoro derivative (S)-13 in good yield.

Illustrative embodiments are described in more detail below in the following numbered paragraphs:

1. A method for preventing or treating skin damage in a radiotherapy subject, comprising topically administering to the subject a composition that includes a therapeutically effective amount at least one targeted nitroxide agent and at least one additional ingredient, wherein the targeted nitroxide agent is selected from:

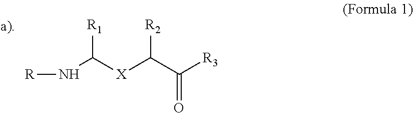

wherein X is one of

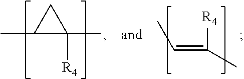

R₁ and R₂ are hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a Cᵣ C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N—O containing group; R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted;
b). a compound having the structure R1-R2-R3 in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; or

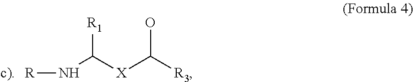

wherein X is one of

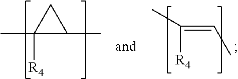

R₁ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted.
2. A method for preventing or treating UV-induced damage in a subject, comprising topically administering to the subject a composition that includes at least one targeted nitroxide agent and at least one additional ingredient, wherein the targeted nitroxide agent is present in the composition in an amount sufficient to achieve a cutaneous cumulative concentration of the targeted nitroxide agent in the subject of 1 pmole/mg to 100 μmole/mg over a 24 hour period after administration, and wherein the targeted nitroxide agent is selected from:

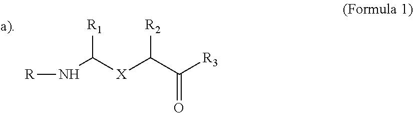

wherein X is one of

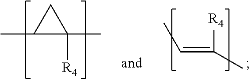

R₁ and R₂ are hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted;
b). a compound having the structure R1-R2-R3 in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; or

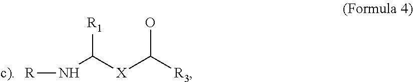

wherein X is one of

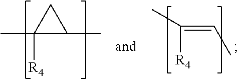

R₁ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted.
3. A composition for topically administering to a subject, wherein the composition comprises 0.1 to 100 mg/ml of at least one targeted nitroxide agent and at least one additional ingredient; the composition is in the form of a suspension, colloid or emulsion; and the composition has a sufficient viscosity that keeps the targeted nitroxide agent in contact with a treated area for a sufficient period of time to allow suitable absorption into the treated area, wherein the targeted nitroxide agent is selected from:

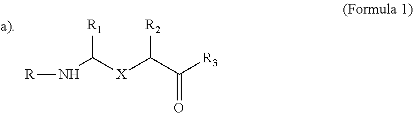

wherein X is one of

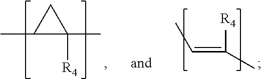

R₁ and R₂ are hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or NO containing group; R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted;
b). a compound having the structure R1-R2-R3 in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; or

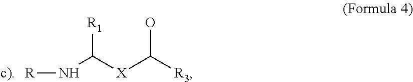

wherein X is one of

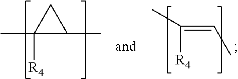

R₁ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted.
4. The method of paragraph 1, wherein the composition is topically administered to the subject 10 minutes to 24 hours after a radiotherapy exposure.
5. The method of paragraph 1, wherein the composition is topically administered to the subject 30 minutes to 2 hours after a radiotherapy exposure.
6. The method of any one of paragraphs 1, 4 or 5, wherein the radiotherapy is cancer radiotherapy.
7. The method of paragraph 6, wherein the composition is topically administered to a post-mastectomy radiotherapy subject.
8. The method of paragraph 1, wherein the composition is topically administered to the subject prior to radiotherapy exposure.
9. The method of any one of paragraphs 1, or 4 to 8, wherein the targeted nitroxide agent is present in the composition in an amount sufficient to achieve a cutaneous cumulative concentration of the targeted nitroxide agent in the subject of I pmole/mg to 100 μmole/mg over a 24 hour period after administration of the composition.
10. The method of any one of paragraphs 1, or 4 to 9, wherein the composition comprises 0.1 to 100 mg/ml of at least one targeted nitroxide agent; the composition is in the form of a suspension, colloid or emulsion; and the composition has a sufficient viscosity that keeps the targeted nitroxide agent in contact with a treated area for a sufficient period of time to allow suitable absorption into the treated area.
11. The method of paragraph 2, wherein UV-induced damage in a subject is photoaging.
12. The method of paragraph 2, wherein the method comprises preventing or treating UVA/UVB-induced carcinogenesis.
13. The method of paragraph 2, wherein the composition is topically administered to the subject 10 minutes to 24 hours after UV exposure.
14. The method of paragraph 2, wherein the composition is topically administered to the subject 30 minutes to 2 hours after UV exposure.
15. The method of paragraph 2, wherein the composition is topically administered to the subject prior to exposure.
16. The method of any one of paragraphs 2, or 11 to 15, wherein the composition comprises 0.1 to 100 mg/ml of at least one targeted nitroxide agent; the composition is in the form of a suspension, colloid or emulsion; and the composition has a sufficient viscosity that keeps the targeted nitroxide agent in contact with a treated area for a sufficient period of time to allow suitable absorption into the treated area.
17. The method of any one of paragraphs 1, 2 or 4-16, the compound having the structure

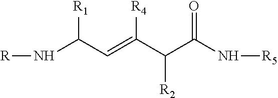

or the structure

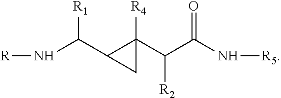

18. The method of any one of paragraphs 1, 2 or 4-16, the compound having the structure

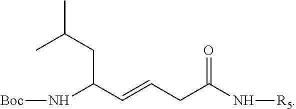

19. The method of any one of paragraphs 1, 2 or 4-16, the compound having the structure

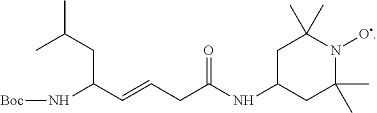

20. The method of any one of paragraphs 1, 2 or 4-16, the compound having the structure

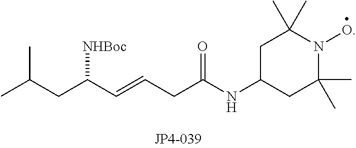

21. The method of any one of paragraphs 1, 2 or 4-16, in which R is Ac, Boc, Cbz, or —P(O)-Ph₂.
22. The method of any one of paragraphs 1, 2 or 4-16, in which R₁, R₂, R₄ and R₆ are independently chosen from hydrogen, methyl, ethyl, propyl, 2-propyl, butyl, t-butyl, pentyl, hexyl, benzyl, hydroxybenzyl, phenyl and hydroxyphenyl.
23. The method of any one of paragraphs claim 1, 2 or 4-16, wherein when X is —CH═CR₄—, R₄ is hydrogen, methyl or ethyl.
24. The method of any one of paragraphs 1, 2 or 4-16, in which R₅ is 2,2,6,6-Tetramethyl-4-piperidine 1-oxyl, 1-methyl azaadamantane N-oxyl, or 1,1,3,3-tetramethylisoindolin-2-yloxyl.
25. The method of any one of paragraphs 1, 2 or 4-16, the compound having the structure:

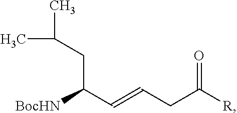

or the structure

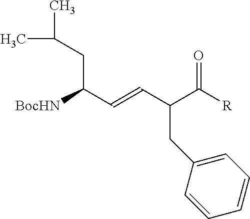

in which R is —NH—R₁, —O—R₁ or —CH₂—R₁, and R₁ is an —N—O., —N—OH or N═O containing group.
26. The method of any one of paragraphs 1, 2 or 4-16, the compound having the structure:

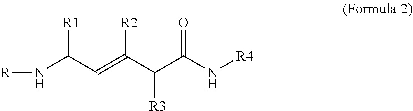

in which R1, R2 and R3 are, independently, hydrogen, C₁-C₆ straight or branched-chain alkyl, or C₁-C₆ straight or branched-chain alkyl including a phenyl (C₆H₅) group that is unsubstituted, methyl-, hydroxyl- or fluoro-substituted; R4 is an —N—O., —N—OH or N═O containing group; and R is —C(O)—R5, —C(O)O—R5, or —P(O)—(R5)₂, wherein R5 is C₁-C₆ straight or branched-chain alkyl, or C₁-C₆ straight or branched-chain alkyl including a phenyl (Ph, C₆H₅) group that is unsubstituted, methyl-, hydroxyl- or fluoro-substituted.
27. The method of any one of paragraphs 1, 2 or 4-16, in which R is Ac, Boc, Cbz, or —P(O)-Ph₂.
28. The method of any one of paragraphs 1, 2 or 4-16 in which R1, R2 and R3 independently are methyl, ethyl, propyl, 2-propyl, butyl, t-butyl, pentyl, hexyl, benzyl, hydroxybenzyl, phenyl and hydroxyphenyl.
29. The method of any one of paragraphs 1, 2 or 4-16, in which R4 is 2,2,6,6-Tetramethyl-4-piperidine 1-methyl azaadamantane N-oxyl), or 1,1,3,3-tetramethyl isoindolin-2-yloxy.
30. The method of any one of paragraphs 1, 2 or 4-16, the compound having a structure chosen from

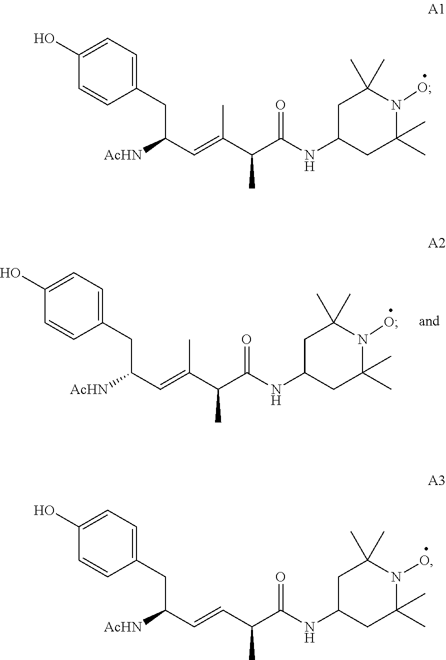

wherein Ac is acetyl.
31. The method of any one of paragraphs 1, 2 or 4-16, the compound having the structure:

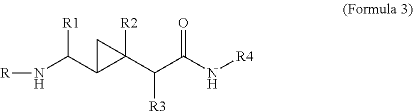

in which R1, R2 and R3 are, independently, hydrogen, C₁-C₆ straight or branched-chain alkyl, or C₁-C₆ straight or branched-chain alkyl including a phenyl (C₆H₅) group that is unsubstituted, methyl-, hydroxyl- or fluoro-substituted; R4 is an —N—O., —N—OH or N═O containing group; R is —C(O)—R5, —C(O)O—R5, or —P(O)—(R5)₂, wherein R5 is C₁-C₆ straight or branched-chain alkyl, or C₁-C₆ straight or branched-chain alkyl including a phenyl (C₆H₅) group that is unsubstituted, methyl-, hydroxyl- or fluoro-substituted.
32. The composition of paragraph 3, further comprising at least one sunscreen agent.
33. The method of paragraph 1, further comprising administering radiotherapy to the subject.

In view of the many possible embodiments to which the principles of the disclosed compositions and methods may be applied, it should be recognized that the illustrated embodiments are only preferred examples and should not be taken as limiting the scope of the invention. Rather, the scope of the invention is defined by the following claims. We therefore claim as our invention all that comes within the scope and spirit of these claims.

What is claimed is:

1. A method for preventing or treating skin damage in a radiotherapy subject, comprising topically administering to the subject a composition that includes a therapeutically effective amount at least one targeted nitroxide agent and at least one additional ingredient, wherein the targeted nitroxide agent is selected from: 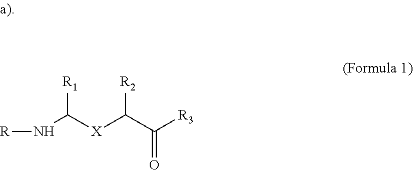
wherein X is one of 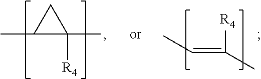
R₁ and R₂ are hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or NO containing group; R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted;
b). a compound having the structure R1-R2-R3 in which R1 and R3 are the same or different and have the structure —R4-R5, in which R4 is a mitochondria targeting group and R5 is —NH—R6, —O—R6 or —CH₂—R6, wherein R6 is an —N—O., —N—OH or NO containing group and R4 and R5 for each of R1 and R3 may be the same or different; and R2 is a linker; or 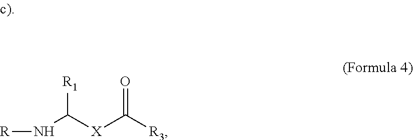
wherein X is one of 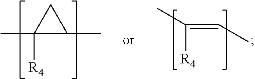
R₁ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted.

2. A method for preventing or treating UV-induced damage in a subject, comprising topically administering to the subject a composition that includes at least one targeted nitroxide agent and at least one additional ingredient, wherein the targeted nitroxide agent is present in the composition in an amount sufficient to achieve a cutaneous cumulative concentration of the targeted nitroxide agent in the subject of 1 pmole/mg to 100 μmole/mg over a 24 hour period after administration, and wherein the targeted nitroxide agent is selected from: 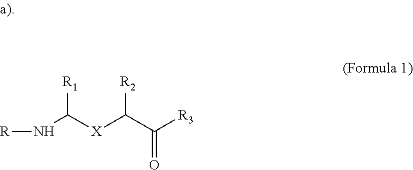
wherein X is one of 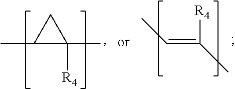
R₁ and R₂ are hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted;
b). a compound having the structure R1-R2-R3 in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; or 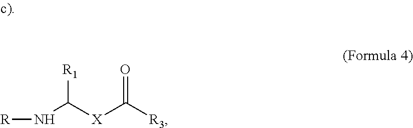
wherein X is one of 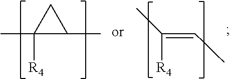
R₁ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted.

3. A composition for topically administering to a subject, wherein the composition comprises 0.1 to 100 mg/ml of at least one targeted nitroxide agent and at least one additional ingredient; the composition is in the form of a suspension, colloid or emulsion; and the composition has a sufficient viscosity that keeps the targeted nitroxide agent in contact with a treated area for a sufficient period of time to allow suitable absorption into the treated area, wherein the targeted nitroxide agent is selected from: 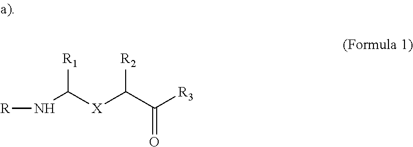
wherein X is one of 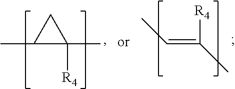
R₁ and R₂ are hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; R is —C(O)—R₅, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted;
b). a compound having the structure R1-R2-R3 in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; or 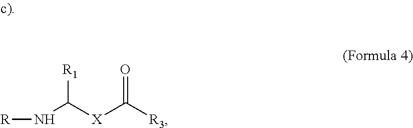
wherein X is one of 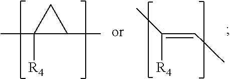
R₁ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted.

4. The method of claim 1, wherein the composition is topically administered to the subject 10 minutes to 24 hours after a radiotherapy exposure.

5. The method of claim 1, wherein the composition is topically administered to the subject 30 minutes to 2 hours after a radiotherapy exposure.

6. The method of claim 1, wherein the radiotherapy is cancer radiotherapy.

7. The method of claim 6, wherein the composition is topically administered to a post-mastectomy radiotherapy subject.

8. The method of claim 1, wherein the composition is topically administered to the subject prior to radiotherapy exposure.

9. The method of claim 1, wherein the targeted nitroxide agent is present in the composition in an amount sufficient to achieve a cutaneous cumulative concentration of the targeted nitroxide agent in the subject of 1 μmole/mg to 100 μmole/mg over a 24 hour period after administration of the composition.

10. The method of claim 1, wherein the composition comprises 0.1 to 100 mg/ml of at least one targeted nitroxide agent; the composition is in the form of a suspension, colloid or emulsion; and the composition has a sufficient viscosity that keeps the targeted nitroxide agent in contact with a treated area for a sufficient period of time to allow suitable absorption into the treated area.

11. The method of claim 2, wherein UV-induced damage in a subject is photoaging.

12. The method of claim 2, wherein the method comprises preventing or treating UVA/UVB-induced carcinogenesis.

13. The method of claim 2, wherein the composition is topically administered to the subject 10 minutes to 24 hours after UV exposure.

14. The method of claim 2, wherein the composition is topically administered to the subject 30 minutes to 2 hours after UV exposure.

15. The method of claim 2, wherein the composition is topically administered to the subject prior to exposure.

16. The method of claim 1, the compound having the structure

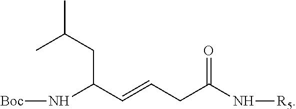

17. The method of claim 1, the compound having the structure

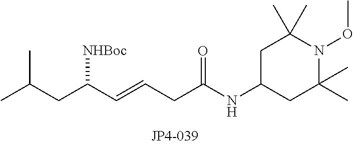

18. The method of claim 1, in which R is Ac, Boc, Cbz, or —P(O)-Ph₂; R₁, R₂, R₄ and R₆ are independently chosen from hydrogen, methyl, ethyl, propyl, 2-propyl, butyl, t-butyl, pentyl, hexyl, benzyl, hydroxybenzyl, phenyl and hydroxyphenyl; and R₅ is 2,2,6,6-Tetramethyl-4-piperidine 1-oxyl, 1-methyl azaadamantane N-oxyl, or 1,1,3,3-tetramethylisoindolin-2-yloxyl.

19. The composition of claim 3, further comprising at least one sunscreen agent.

20. The method of claim 1, further comprising administering radiotherapy to the subject.

21. A method for preventing or treating ionizing radiation-induced damage in a subject, comprising topically administering to the subject a composition that includes at least one targeted nitroxide agent and at least one additional ingredient, wherein the targeted nitroxide agent is present in the composition in an amount sufficient to achieve a cutaneous cumulative concentration of the targeted nitroxide agent in the subject of 1 pmole/mg to 100 μmole/mg over a 24 hour period after administration, and wherein the targeted nitroxide agent is selected from: 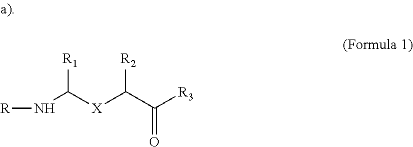
wherein X is one of 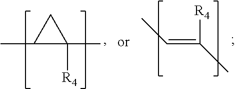
R₁ and R₂ are hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted;
b). a compound having the structure R1-R2-R3 in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; or 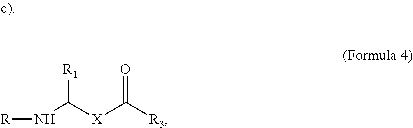
wherein X is one of 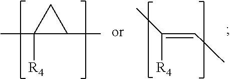
R₁ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₄ is hydrogen, C₁-C₆ straight or branched-chain alkyl, or a C₁-C₆ straight or branched-chain alkyl further comprising a phenyl (C₆H₅) group, that is unsubstituted or is methyl-, hydroxyl- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl- or fluoro-substituted.

22. A method for preventing or treating skin damage in a radiotherapy subject, comprising topically administering to the subject a composition that includes a therapeutically effective amount at least one targeted nitroxide agent and at least one additional ingredient, wherein the targeted nitroxide agent has a structure of: 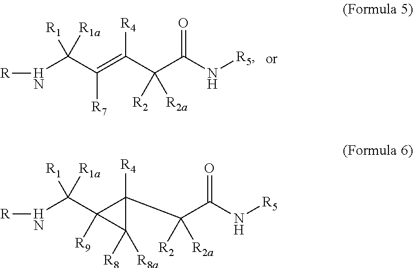
wherein R₁, R₁ₐ, R₂, and R₂ₐ are independently hydrogen, a halo, C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₄ is hydrogen, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or a C₁-C₆ straight or branched-chain alkyl further comprising one or more (C₆H₅) groups that are independently unsubstituted, or methyl-, ethyl-, hydroxyl- or halo-substituted; R₇, R₈, R₈ₐ, and R₉ are independently H, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted, provided that at least one of R₁, R₁ₐ, R₂, R₂ₐ, or R₇ is not H.

23. A method for preventing or treating UV-induced damage in a subject, comprising topically administering to the subject a composition that includes at least one targeted nitroxide agent and at least one additional ingredient, wherein the targeted nitroxide agent is present in the composition in an amount sufficient to achieve a cutaneous cumulative concentration of the targeted nitroxide agent in the subject of 1 pmole/mg to 100 μmole/mg over a 24 hour period after administration, and wherein the targeted nitroxide agent has a structure of 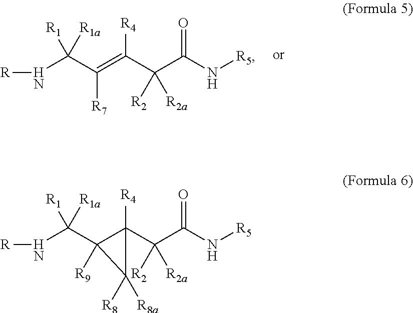
wherein R₁, R₁ₐ, R₂, and R₂ₐ are independently hydrogen, a halo, C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₄ is hydrogen, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or a C₁-C₆ straight or branched-chain alkyl further comprising one or more (C₆H₅) groups that are independently unsubstituted, or methyl-, ethyl-, hydroxyl- or halo-substituted; R₇, R₈, R₈ₐ, and R₉ are independently H, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted, provided that at least one of R₁, R₁ₐ, R₂, R₂ₐ, or R₇ is not H.

24. A composition for topically administering to a subject, wherein the composition comprises 0.1 to 100 mg/ml of at least one targeted nitroxide agent and at least one additional ingredient; the composition is in the form of a suspension, colloid or emulsion; and the composition has a sufficient viscosity that keeps the targeted nitroxide agent in contact with a treated area for a sufficient period of time to allow suitable absorption into the treated area, wherein the targeted nitroxide agent has a structure of 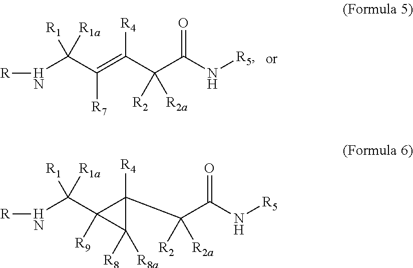
wherein R₁, R₁ₐ, R₂, and R₂ₐ are independently hydrogen, a halo, C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₄ is hydrogen, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or a C₁-C₆ straight or branched-chain alkyl further comprising one or more (C₆H₅) groups that are independently unsubstituted, or methyl-, ethyl-, hydroxyl- or halo-substituted; R₇, R₈, R₈ₐ, and R₉ are independently H, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted, provided that at least one of R₁, R₁ₐ, R₂, R₂ₐ, or R₇ is not H.

25. A method for preventing or treating ionizing radiation-induced damage in a subject, comprising topically administering to the subject a composition that includes at least one targeted nitroxide agent and at least one additional ingredient, wherein the targeted nitroxide agent is present in the composition in an amount sufficient to achieve a cutaneous cumulative concentration of the targeted nitroxide agent in the subject of 1 pmole/mg to 100 mmole/mg over a 24 hour period after administration, and wherein the targeted nitroxide agent has a structure of 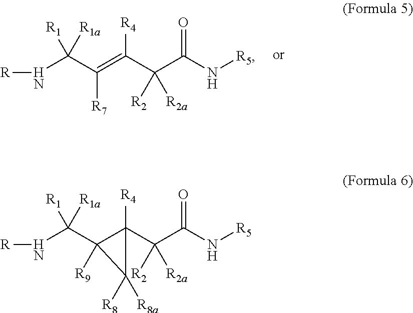
wherein R₁, R₁ₐ, R₂, and R₂ₐ are independently hydrogen, a halo, C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₄ is hydrogen, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or a C₁-C₆ straight or branched-chain alkyl further comprising one or more (C₆H₅) groups that are independently unsubstituted, or methyl-, ethyl-, hydroxyl- or halo-substituted; R₇, R₈, R₈ₐ, and R₉ are independently H, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted, provided that at least one of R₁, R₁ₐ, R₂, R₂ₐ, or R₇ is not H.
